(19) United States
(12) Patent Application Publication
(10) Pub. No.: US 2025/0064932 A1
(43) Pub. Date: Feb. 27, 2025

(54) GLYCO-ENGINEERED CAR-T CELLS

(71) Applicants: VIB VZW, Gent (BE); Universiteit Gent, Gent (BE)

(72) Inventors: Nico Callewaert, Nevele (BE); Nele Festjens, Kluisbergen (BE); Elien De Bousser, Gent (BE)

(21) Appl. No.: 18/720,885

(22) PCT Filed: Dec. 16, 2022
(86) PCT No.: PCT/EP2022/086474

§ 371 Date: Jun. 17, 2024

(30) Foreign Application Priority Data
Dec. 17, 2021 (EP) 21215482.7

(51) Int. Cl. A61K 39/00; A61P 35/00; C12N 5/0783; C12N 9/10; C12N 9/22; C12N 15/11; C12N 15/90

(52) U.S. Cl. CPC A61K 39/4637; A61K 39/4611; A61K 39/4631; A61P 35/00; C12N 5/0636; C12N 9/1051; C12N 9/22; C12N 15/11; C12N 15/907; C12Y 204/01155; C12N 2310/20

(57) ABSTRACT

The invention relates to the field of CAR-T cell immunotherapy, more specifically the invention relates to production and uses of glyco-engineered CAR-T cells for the improvement of immunotherapy compositions for treatment of cancer, more specifically of solid tumors. The invention specifically relates to human CAR-T cells with a mutated MGAT5 gene, as to provide for surface glycan structures devoid of tetra-antennary N-glycans, which results in a sustained memory when applied in immunotherapy, to cure cancer, reduce (recurrent) tumor growth and tumor burden, as well as to prevent relapse. The invention further relates to methods for manufacturing of those CAR-T cells, wherein addition of low amounts of DMSO during activation and expansion ex vivo skews T cell populations to a more predominant memory phenotype, thereby providing for improved glycol-engineered CAR-T cell compositions for adoptive T cell transfer.

### CROSS REFERENCE TO RELATED APPLICATIONS

This application is a national phase entry under 35 U.S.C. § 371 of International Patent Application PCT/EP2022/086474, filed Dec. 16, 2022, designating the United States of America and published in English as International Patent Publication WO2023/111322 on Jun. 22, 2023, which claims the benefit under Article 8 of the Patent Cooperation Treaty to European Patent Application Serial No. 21215482.7, filed Dec. 17, 2021, the entireties of which are hereby incorporated by reference.

### INCORPORATION BY REFERENCE

The ST.26 XML Sequence listing named “Sequence Listing ST26”, created on Dec. 16, 2022, and having a size of 8,192 bytes, is hereby incorporated herein by this reference in its entirety.

### FIELD OF THE INVENTION

The invention relates to the field of CAR-T cell immunotherapy, more specifically the invention relates to production and uses of glyco-engineered CAR-T cells for the improvement of immunotherapy compositions for treatment of cancer, more specifically of solid tumors. The invention specifically relates to human CAR-T cells with a mutated MGAT5 gene, as to provide for surface glycan structures devoid of tetra-antennary N-glycans, which results in a sustained memory when applied in immunotherapy, to cure cancer, reduce (recurrent) tumor growth and tumor burden, as well as to prevent relapse. The invention further relates to methods for manufacturing of those CAR-T cells, wherein addition of low amounts of DMSO during activation and expansion ex vivo skews T cell populations to a more predominant memory phenotype, thereby providing for improved glycol-engineered CAR-T cell compositions for adoptive T cell transfer.

### INTRODUCTION

Immunotherapy with T cells which are genetically modified to express chimeric antigen receptors (CARs), that target tumor-associated molecules, has shown impressive efficacy in several malignancies.

The advent of second-generation CAR-T cells, in which activating and costimulatory signaling domains are combined, has led to encouraging results in patients with chemo-refractory B cell malignancies^{42,43} However, the translation of this clinical success to the treatment of solid tumors requires that multiple obstacles are overcome. In general, generation of a robust and stable population of T cells is required that are able to infiltrate the tumor and escape the immunosuppressive effect of the tumor microenvironment (TME). Further issues in CAR-T cell therapy include antigen escape, CAR-T cell toxicity and the relatively high occurrence of tumor relapse.

Cell surface glycosylation plays an important role in the interaction of human T cells with tumor cells, and often contributes to escape mechanisms adopted by the tumor to evade T cell anti-tumor immunity. For example, the expression of immune checkpoint inhibitors such as PD-1 and CTLA-4 is tuned by glycosylation⁴⁴⁻⁴⁶. Furthermore, glycan binding proteins such as Galectins can be overexpressed in the TME leading to tolerogenic signaling and immune suppression^{47,48}. Manipulations of the surface glycome of T cells and/or tumor cells however affect the entire glycome in most cases, not restricted to one particular glycosylated molecule.

N-acetylglucosaminyltransferase V (MGAT5) initiates P1,6-GlcNAc N-glycan branching (FIG. 1), and is involved in multiple aspects of T cell activation, since MGAT5 deficient mice were observed to be more susceptible to experimental autoimmune diseases³, and the deficiency of MGAT5 was shown to markedly increase TCR clustering and signaling at the immune synapse, resulting in a lower T cell activation threshold and increased incidence of autoimmune disease in vivo and in human³. Moreover, mgat5 and pdia3 ko mutants revealed glyco-engineered CD8+ T cells, which resulted in enhanced survival of glioblastoma (GBM)-bearing mice⁶.

The immune cell chassis used to express a CAR is most commonly a population of T-cells derived from the peripheral blood. After ex vivo transduction, the CAR-T cell culture requires expansion, a phase during which it is desirable to keep the CAR-T cells in a more naïve condition, have less effector cells and obtain a larger proportion of viable cells. Moreover, the naïve/memory phenotype of CAR-T cells is advantageous for engrafting upon adoptive T cell transfer (ACT) to the patient.

Dimethyl sulfoxide (DMSO) is a small amphipathic molecule composed of a highly polar domain characterized by a sulfinyl group and two nonpolar methyl groups. For this reason, it is able to solubilize both polar and nonpolar substances and transpose hydrophobic barriers. DMSO is extensively used in biological and medical research to dissolve pharmacological substances such as drugs, as well as a solvent for peptides and proteins with therapeutic applications or for immune functional assays¹⁵. DMSO also exerts anti-inflammatory, reactive oxygen species (ROS) scavenger and immunomodulatory effects and therefore exhibits therapeutic potential for the treatment of several inflammatory diseases. In many countries, DMSO is prescribed for a variety of diseases including arthritis and interstitial cystitis, and to treat symptoms such as pain, inflammation and intercranial pressure¹⁶. This makes DMSO one of the most studied, but least understood pharmaceutical agents. Further, DMSO is routinely used as a cryoprotectant for long-term cell freezing and other applications of this molecule in cell culture are broad. The commercial preparations of FDA-approved formulations of CAR-T cell compositions, Kymriah and Yescarta, also contain 5% DMSO for the cryopreservation. Different cell types respond very differently to varying DMSO concentrations and stimulatory conditions¹⁷⁻²¹. Partly because of this, the described effects of DMSO on cell biology have been a topic of ongoing debate. Awan et al recently reviewed the biological impact of the use of DMSO, with reference to clinical applications²². In the literature, the hemolytic concentration of DMSO is controversial, varying largely according to the experimental conditions adopted^{23,24}. DMSO toxicity is mostly explained by its effect on the physical properties of the phospholipids in membranes. It can transpose hydrophobic barriers such as the plasma membrane allowing pore formation, contributing to decreased membrane selectivity and increased cell permeability²⁵. However, some concentrations of DMSO might modify the cellular activation in vitro.

So, the preparation and manufacturing process of CAR-T cell compositions for use in immunotherapy require properties and memory phenotypes that are beneficial for ACT and engrafting, and ultimately allow for sustainable efficacy to prevent or reduce recurrence. So, there is still a need to improve clinical efficacy by CAR-T cell engineering for haematological malignancy as well as solid tumor treatment.

### SUMMARY OF THE INVENTION

The present invention is based on the study of the impact of cell surface glycosylation engineering on cytotoxic T cell functionality, in both in vitro and in vivo anti-tumor models, with a focus on the MGAT5 glyco-engineering, using a CRISPR-Cas9 strategy to generate (CAR-) T cells lacking MGAT5 expression. This study made use of MGAT5 mutant glyco-engineered CD70 nanoCAR-T cells, which were shown to impact the cytotoxic potential. In vivo anti-tumor functionality of the MGAT5 KO CD70 nanoCAR-T cells was shown on two tumor cell lines (AML and adenocarcinoma cells). To eliminate tumor cells, T cells must not only persist, but sustain cytolytic and proliferative function, eluding the inhibitory signals encountered in the tumor microenvironment. The potential of glycosylation engineering by targeting MGAT5 to alter T cell survival, proliferation and differentiation of the cells was explored on both purified CD8+ T cells and CD70 nanoCAR-engineered T cells to check whether the described glyco-engineering strategy could endow therapeutic cells with new favorable properties and functions. Using flow cytometry-based immunophenotyping in human tumor xenograft models in NOD scid gamma (NSG) immunodeficient mice, the (long-term) CD70 nanoCAR-T cell efficacy was analyzed upon a primary and secondary tumor challenge. Surprisingly, clearly upon secondary challenge, inhibition of secondary tumor growth and lowered secondary tumor burden was observed in mice previously treated with MGAT5 KO CD70 nanoCAR-T cells as compared to the untreated and mock-engineered CD70 nanoCAR-treated group, as well as an improvement in controlling primary tumor growth and development for MGAT5 KO CD70 nanoCAR-T cells.

In a first aspect of the invention, a CAR-T cell or CAR-T cell composition, preferably originating from human T cells, with at least one mutation in the N-acetylglucosaminyltransferase V (MGAT5) gene, providing for a MGAT5 knock-out glyco-engineered T cell is described. Said human CAR-T cell may be obtainable by using a CRISPR/Cas gene editing approach, resulting in the MGAT5 knock-out human CAR-T cell. In one embodiment, said MGAT5 knock-out human CAR-T cell is devoid of tetra-antennary N-glycans at its cell surface. Said cells contain a reduced surface N-glycan β-1,6-branching and subsequent lack of elongation of this branch with poly-LacNAc modifications as compared to the CAR-T cells that are not mutated in MGAT5.

In a further aspect, the MGAT5 glyco-engineered human CAR-T cell is for use as a medicine, more specifically for use in treatment of cancer. A specific embodiment relates to said MGAT5 knock-out human CAR-T cells or composition for use in treatment of a haematological malignancy as well as treatment of a solid tumor. A further specific embodiment The MGAT5 mutated human CAR-T cell for use in treatment of cancer, wherein said treatment using the human MGAT5 knockout CAR-T cells results in sustained efficacy, thereby being capable of curing (primary) tumors, and/or preventing, inhibiting, or blocking relapse of cancer in said treated subject. More specifically, treatment using the MGAT5 mutated human CAR-T cell, prevents, inhibits, blocks or at least reduces (recurrent) tumor growth and tumor burden in a subject, as compared to a subject treated with a human CAR-T cell that is different in that it is not an MGAT5 mutated CAR-T cell.

A further aspect of the invention relates to methods for producing MGAT5 mutated CAR-T cells, and additionally applies a low amounts of DMSO in the culture medium of CAR-T cells during the stimulation and expansion stage since this was shown to lead to more viable cells, a larger proportion of central memory cells, and a decrease in naïve and effector cells, which may have a beneficial effect on engrafting of the cells in a patient upon ACT. It seems that both, the glyco-engineering and the production of CAR-T cells, as described herein, improve the quality of the CAR-T cell population for immunotherapy, since the memory phenotype and sustainable effect show an improvement in CAR-T cell quality and thus in persistence and efficacy in a patient.

So another aspect relates to a method to produce an MGAT5 CAR-T cell composition with a predominant memory phenotype, for improved engrafting, comprising the steps of:
(a) harvesting or isolating T cells from a primary sample, such as a blood sample obtained from a subject,
(b) incubating said isolated T cell population of step a) under stimulating conditions in a suitable culture medium comprising 0.3-1.2% (v/v) DMSO, thereby generating a stimulated T cell population, and wherein the harvested T cell population comprises a plurality of cells with a concentration of at least 1×10⁶ cells/mL,
(c) engineering the stimulated T cell composition by contacting the cells with an agent comprising a polynucleotide encoding the recombinant receptor, as to provide for a CAR-T engineered cell composition, wherein a mutation in the T cells is introduced in the MGAT5 gene,
(d) expansion of the stimulated (CAR-)T cell population over a period in a suitable cultivation medium comprising 0.3-1.2% (v/v) DMSO.

Said method provides for a glycol-engineered CAR-T cell composition or population with a predominant memory phenotype, which may be defined as an increase in the fraction of central memory CAR-T cells within said CAR-T cell composition. Another embodiment relates to said method to produce a CAR-T cell composition wherein the expansion in step d) is over a period of time of at least 5 days.

In a further embodiment, said ‘contacting’ in step (c), is performed by transduction or by transfection with a vector, and wherein the vector may be a viral vector. Alternatively, said ‘contacting’ in step (c), is performed by genetically modifying or introducing the chimeric antigen receptor, optionally using CRISPR/Cas gene editing, or electroporation, or transposon transfection, or any other technology known to the skilled artisan.

In a specific embodiment, said introduction of the mutation in MGAT5 is performed using CRISPR/Cas gene editing, as known to the skilled person.

An alternative embodiment relates to a method to produce a CAR-T cell composition wherein MGAT5 is mutated, with a predominant memory phenotype, comprising the steps of:
(a) harvesting or isolating T cells from a primary sample, such as a blood sample obtained from a subject,
(b) incubating said T cell population of step a) under stimulating conditions in a suitable culture medium comprising 0.3-1.2% (v/v) DMSO, thereby generating a stimulated T cell population, and wherein the T cell population comprises a plurality of cells with a concentration of at least 1×10⁶ cells/mL, and
(b-bis) engineering the stimulated T cell composition by contacting the cells with an agent comprising a polynucleotide encoding the recombinant receptor, as to provide for a CAR-T engineered cell composition, and
(c) expansion of the stimulated T cell population over a period in a suitable cultivation medium comprising 0.3-1.2% (v/v) DMSO,
wherein a further step (b-tris) which is performed after step b), and before, after or simultaneously with step (b-bis) comprises: introducing a mutation in the Mgat5 gene of the T cells, optionally using CRISPR/Cas gene editing.

A further embodiment described herein relates to the use of the method as described herein to prepare a (CAR-)T cell composition for adoptive cell transfer to a subject.

A further aspect relates to the (CAR-) T cell composition obtainable by any of the methods as described herein, or more specifically the MGAT5 mutated CAR-T cell composition obtainable by the methods described herein, or even more specifically the human (CAR-) T cell composition or human MGAT5 mutated (CAR-)T cell composition obtainable by the method described herein.

In another aspect, the invention relates to the CAR-T cell composition or the MGAT5 mutated CAR-T cell composition obtainable by the methods as described herein, for use in treatment of cancer.

A further specific embodiment relate to the MGAT5 mutated CAR-T cell composition obtainable by the methods described herein, for use in treatment of cancer, wherein the treatment prevents, blocks, and/or inhibits or at least reduces relapse, primary and/or recurrent tumor development, growth and/or primary and/or recurrent tumor burden.

A final aspect relates to a pharmaceutical composition which comprises any of said human (CAR-)T cells or cell compositions described herein. And a final embodiment relates to said pharmaceutical compositions for use in treatment of cancer.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIG. 1. Pathway of N-glycan branching. N-glycan branching is achieved through a series of mannosidase (M) and mannosylglycoprotein-N-acetylglucosaminyltransferase (MGAT)-mediated reactions. The glyco-gene product of interest MGAT5 is marked in bold. This branching primarily occurs in the medial-Golgi compartment. In later compartments, branched glycans are acted upon by other enzymes including Gal-, GlcNAc-, sialyl- and fucosyltransferases to result in complex glycans.

FIGS. 2A-2C. MGAT5 glyco-gene editing and CD70 nanoCAR engineering. FIG. 2A. Experimental timeline. FIG. 2B. Mean editing efficiency as % insertions/deletions (indels) obtained for the MGAT5 locus. FIG. 2C. Mean transduction efficiency as % GFP expressing cells for the different engineering conditions.

FIGS. 3A and 3B. Profiling of alterations in cell surface glycosylation upon MGAT5 KO in CD70 nanoCAR-T cells. FIG. 3A. Lectin staining. 2×10⁵ mock engineered (green) or MGAT5 KO (orange) CAR-T cells were collected, stained with fixable viability dye eFI780 and biotinylated lectin followed by secondary staining with PE-coupled neutravidin. Analysis was done by flow cytometry and graphs show the lectin binding signals after gating on viable cells. Representative results are shown from multiple engineering experiments performed on T cells from independent blood donors. FIG. 3B. DSA-FACE profiling. Schematic representation of the sample preparation and DSA-FACE profiles of the cell-surface glycome of mock engineered and MGAT5 KO CAR-T cells. Sialidase digest was performed on the samples prior to the analysis. The major pairs of peaks in the profile are annotated P1-P6.

FIGS. 4A-4C. Characterization of the THP-1 and SKOV-3 cell lines used as target cells. FIG. 4A. CD70 antigen expression on the THP-1 and SKOV-3 target cell lines was evaluated by flow cytometry. Jurkat cells were included as negative control. Non-transduced control (NTC) and CD70 nanoCAR expressing human T cells were included to check for auto-antigen expression. FIG. 4B. Evaluation of the secretion of Galectins by the tumor cell lines under study. Secretion and cell surface binding of Galectin-1 and Galectin-3 by different cell types was assessed by flow cytometry. Jurkat cells were included as negative controls. As a control, lactose was used as competitive inhibitor to reduce cell surface galectin binding. Additionally, cells were incubated with recombinant galectins as positive control. The secretion and cell surface binding of Galectin-1 and Galectin-3 on THP-1 and SKOV-3 cells are summarized in the overlaid histograms. Representative results from two independent experiments are shown. Different conditions are indicated by numbers. FIG. 4C. Representative images of an FFPE tumor sample expressing Galectin-1 (left) and Galectin-3 (right) at a 25× magnification (red) with DAPI nuclear counterstain (blue). Scalebar is 50 μm.

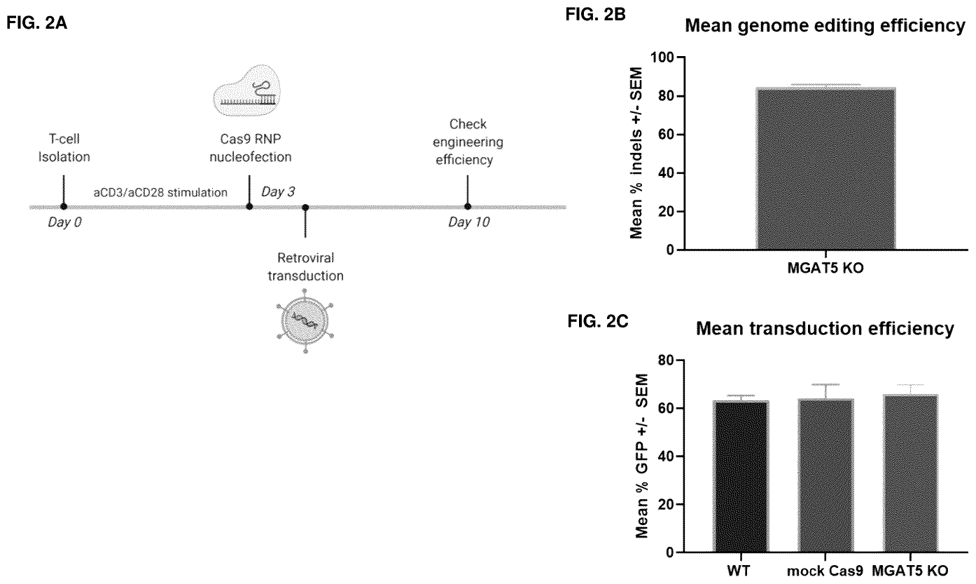

FIG. 2. Effect of MGAT5 KO on CD70 nanoCAR viability and subset distribution at day 13 of in vitro culturing.

FIGS. 6A and 6B. The impact of glyco-engineering of MGAT5 on in vitro CD70 nanoCAR cytokine production. Cytokine production of glyco-engineered CD70 nanoCAR-T cells was evaluated by intracellular staining after co-incubation with SKOV-3 and THP-1 target cell lines for 16 hours. Unstimulated cells were included as negative control (−) while Immunocult stimulation was included as positive control (+). Technical duplicates were analyzed. Mean percentages of TNF-α, IFN-γ and IL-2 positive CD4⁺ (FIG. 6A) or CD8⁺ T cells (FIG. 6B) are shown. Error bars represent standard deviations. The data shown is representative of two independent experiments performed with different donors.

FIGS. 7A and 7B. The impact of glyco-engineering in MGAT5 on in vitro CD70 nanoCAR cytotoxic potential. Glyco-engineered CD70 nanoCAR-T cells were incubated at different effector to target THP-1 cell ratios in duplicate and cell numbers were analyzed over a time period of 14 days. A second challenge with THP-1 cells was added at day 7. Error bars represent the standard deviation on the mean cell number. Data shown are representative for two independent experiments performed on different blood donors. FIG. 7A. results for E/T ratio of 0.15 corresponding to the co-culture of 20 000 THP-1 cells with 3 000 glyco-engineered CD70 nanoCAR-T cells. FIG. 7B. Results for E/T ratio of 0.015 corresponding to the co-culture of 20 000 THP-1 cells with 300 CD70 nanoCAR-T cells.

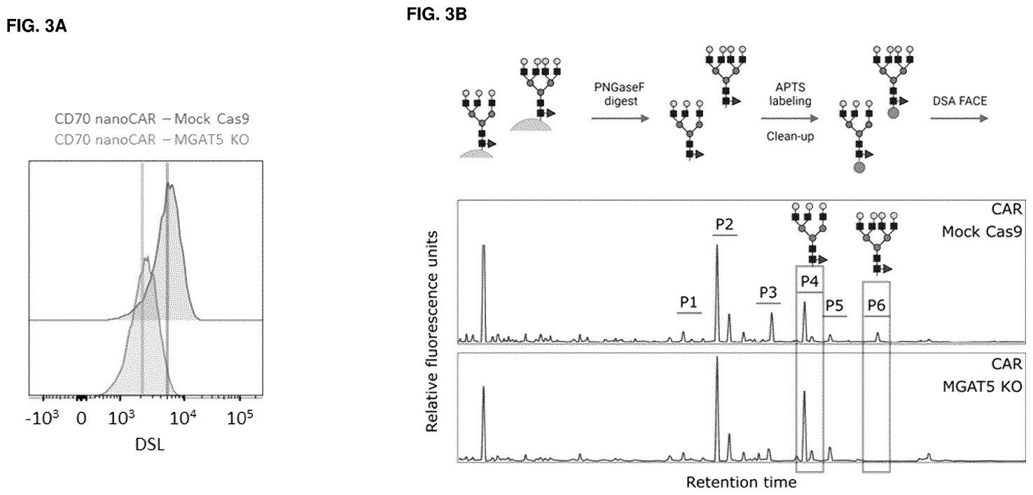

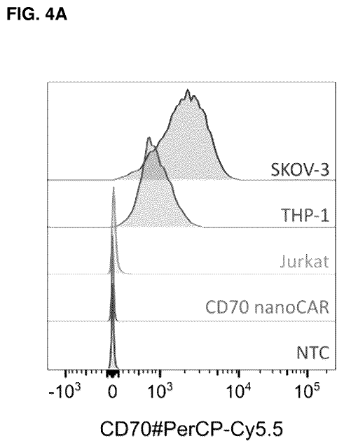

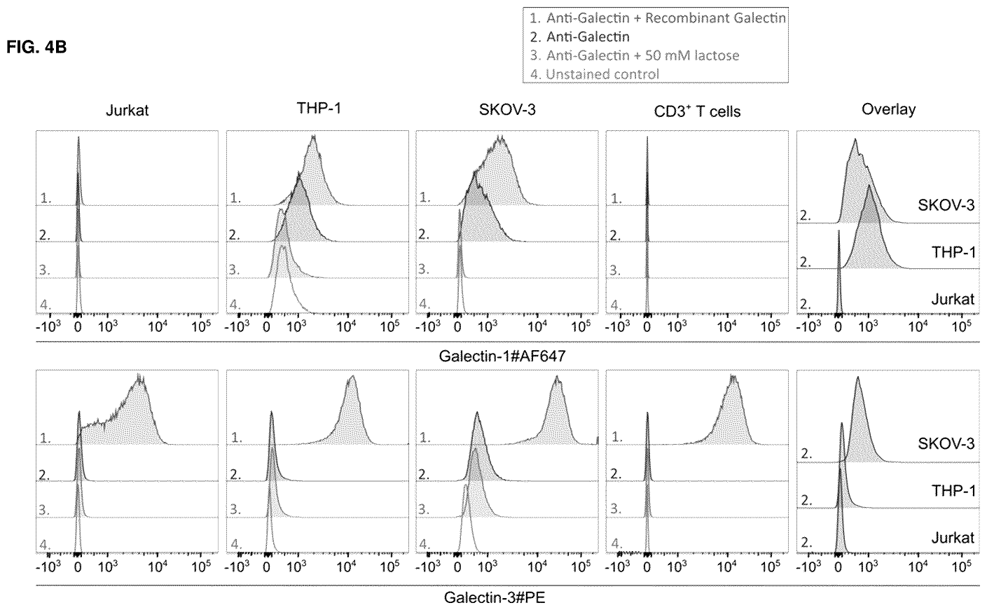

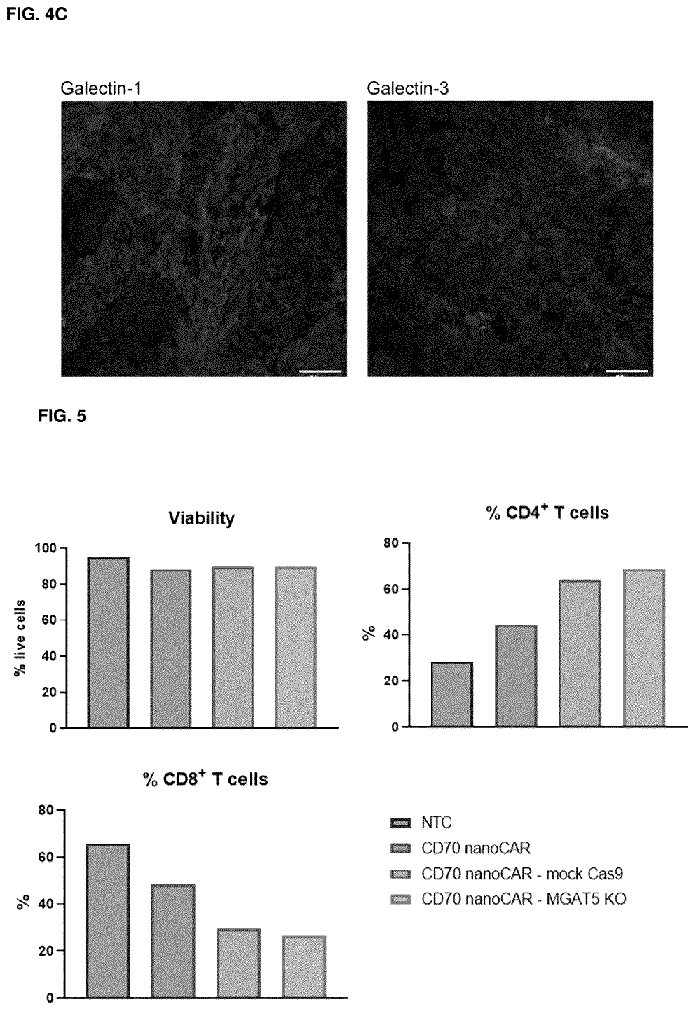

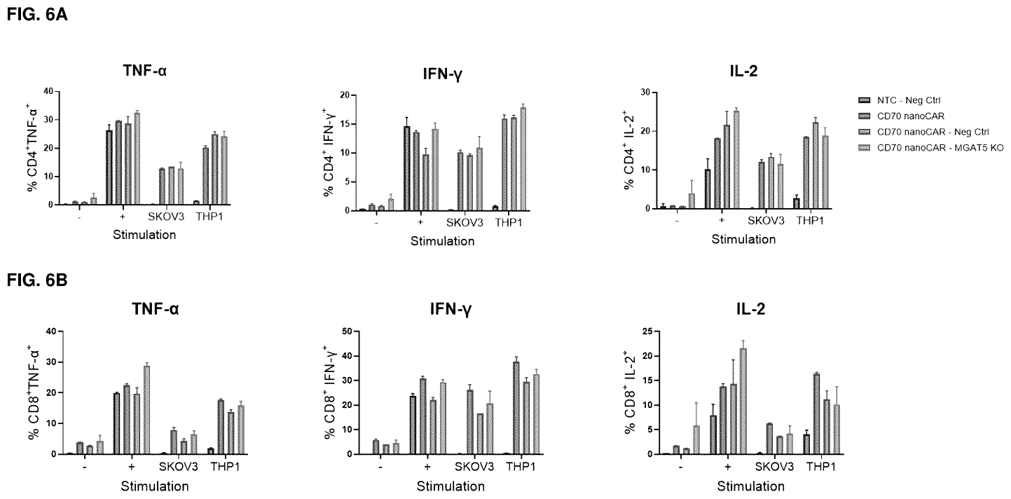

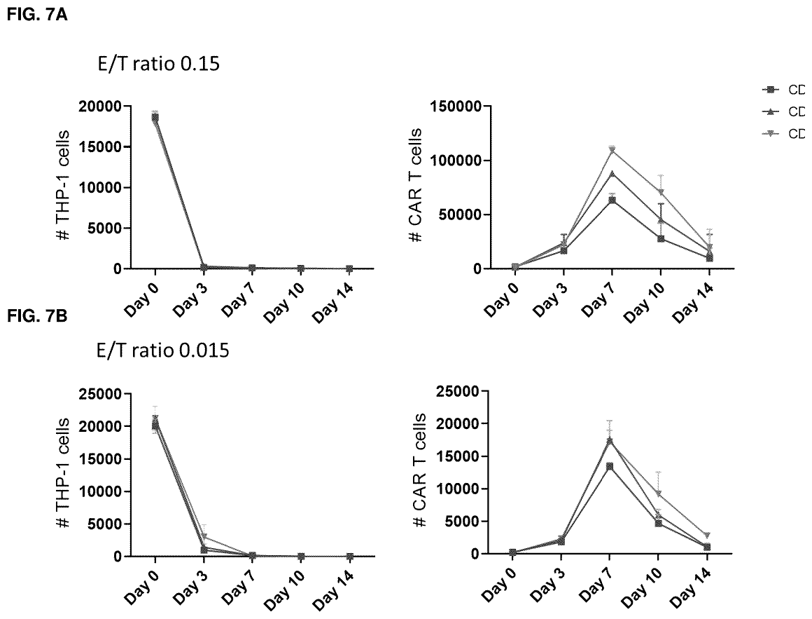

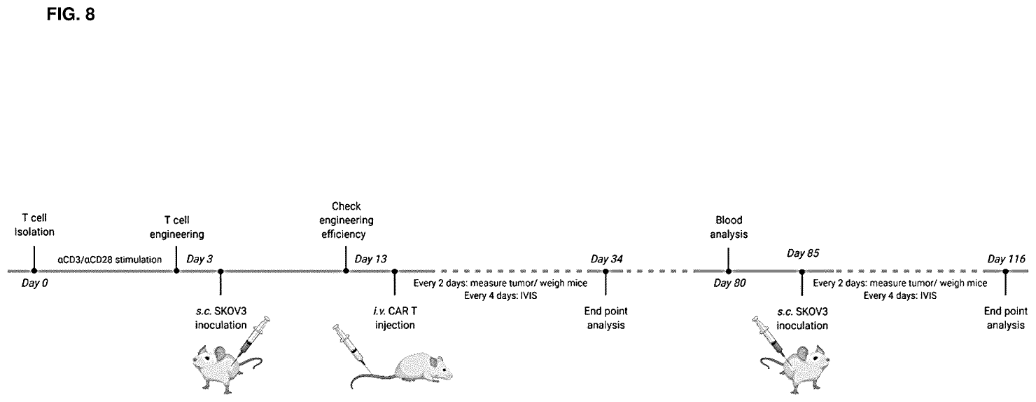

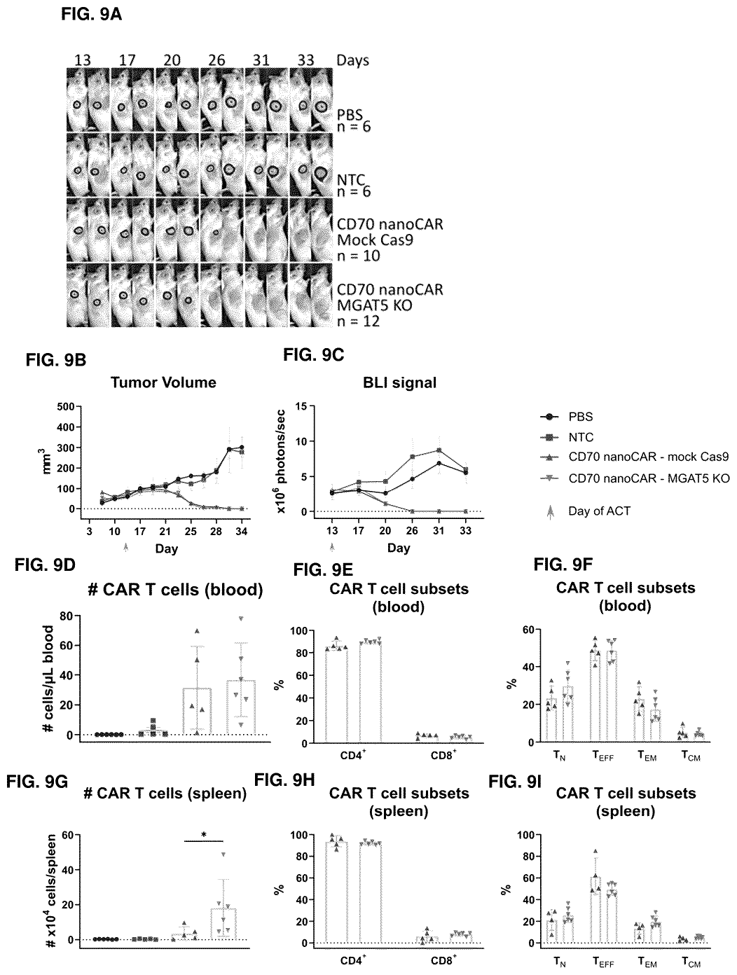

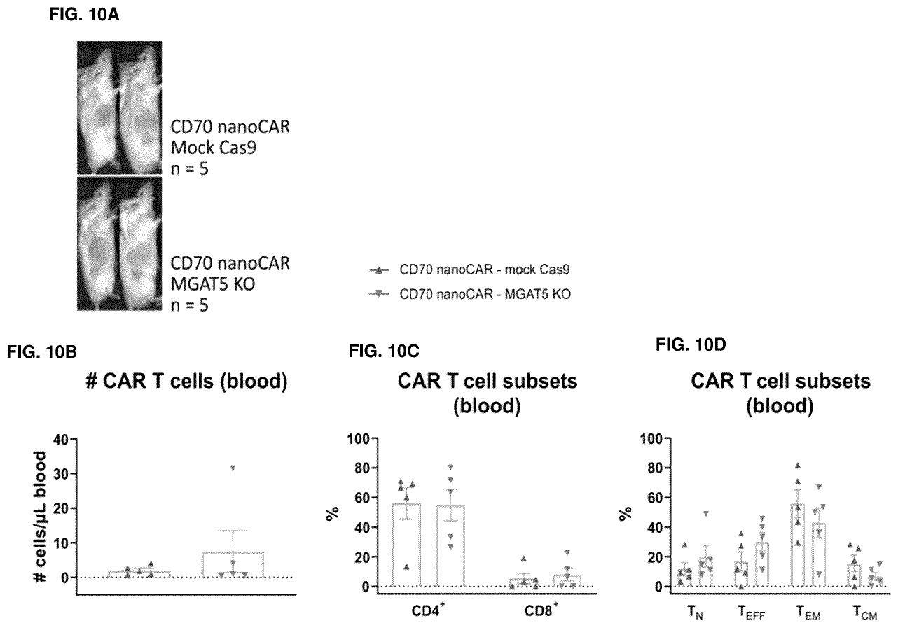

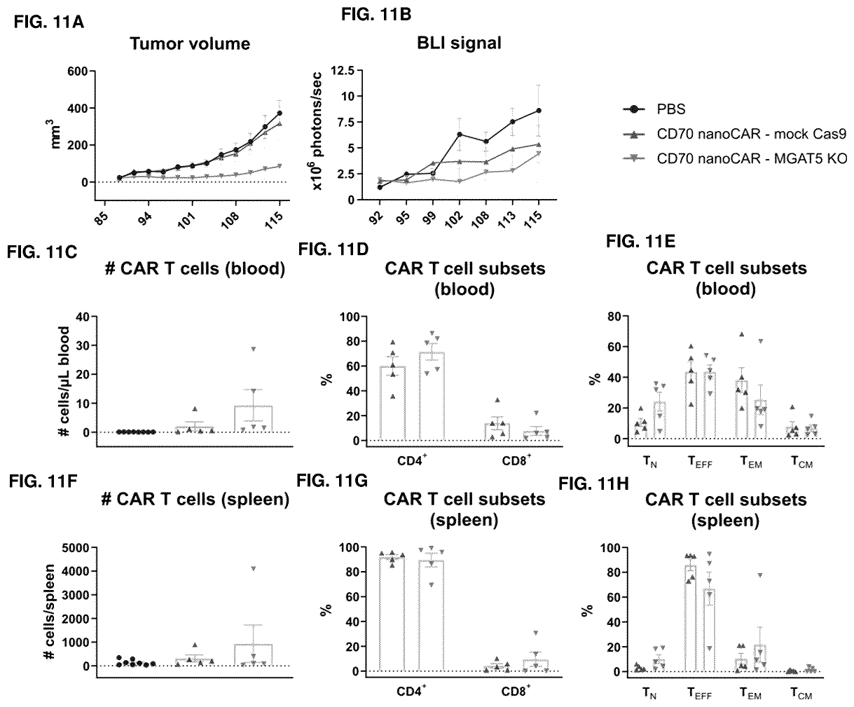

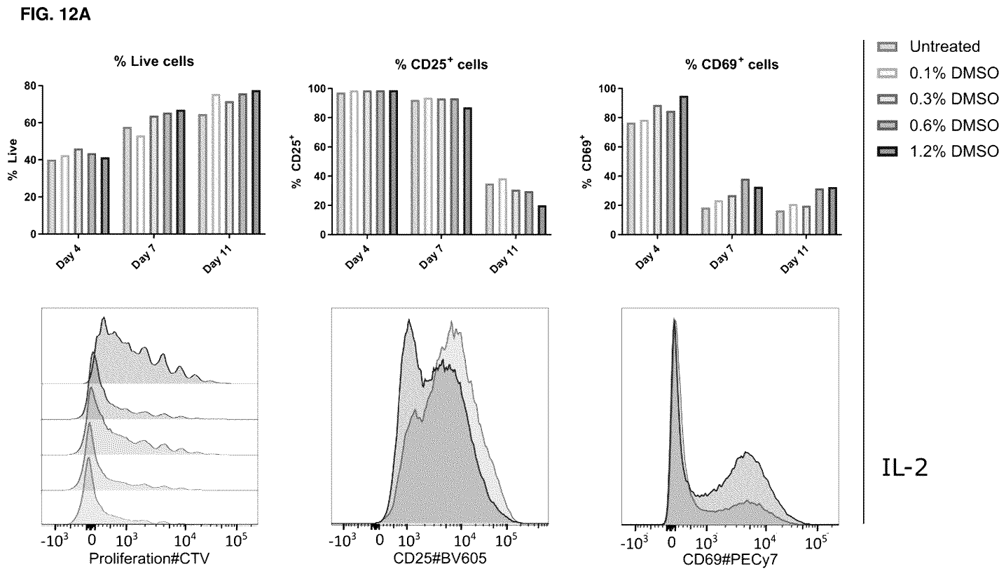

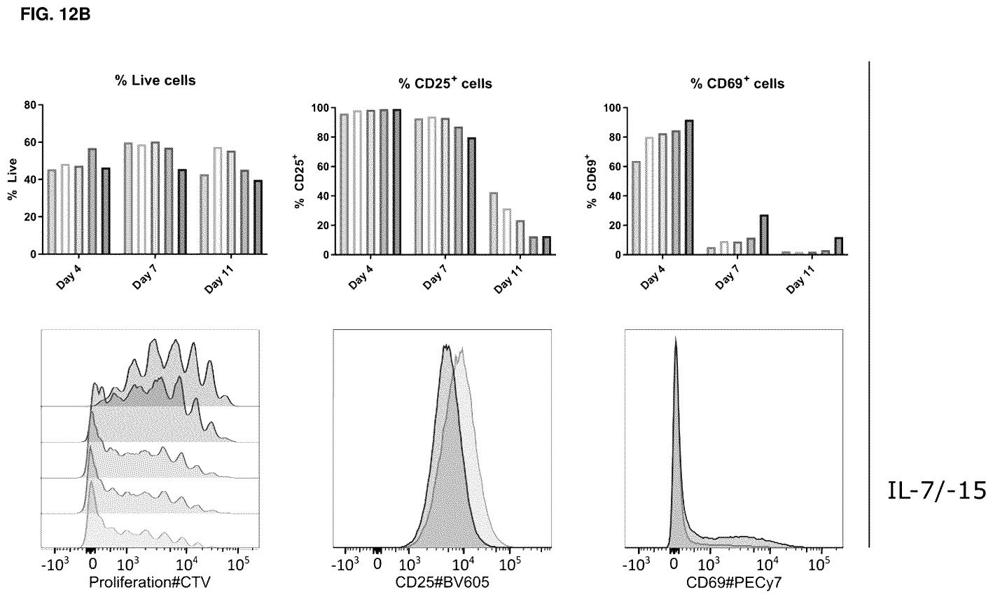

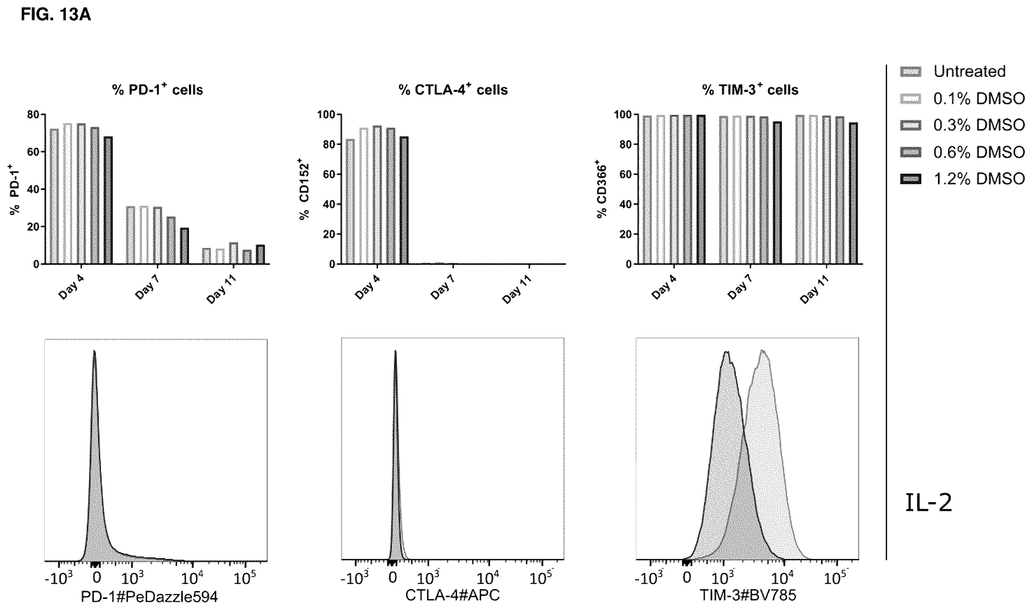

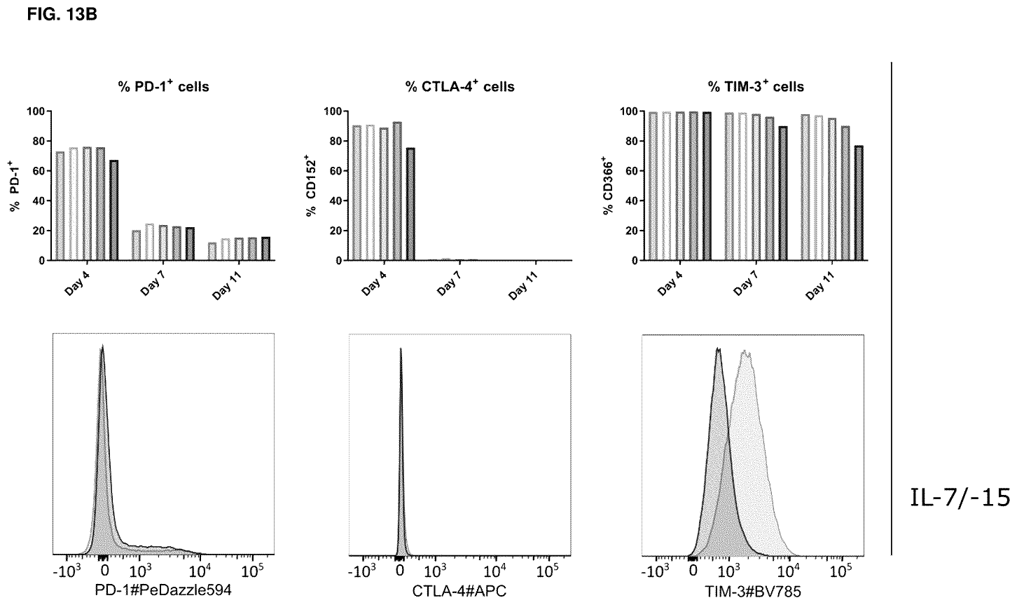

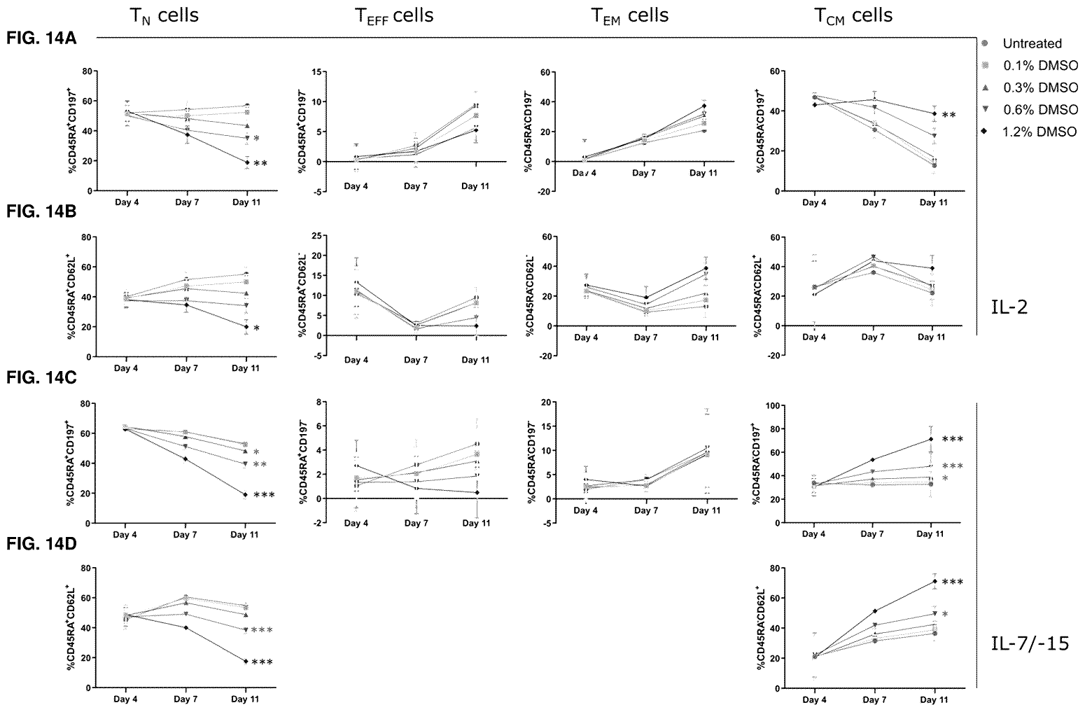

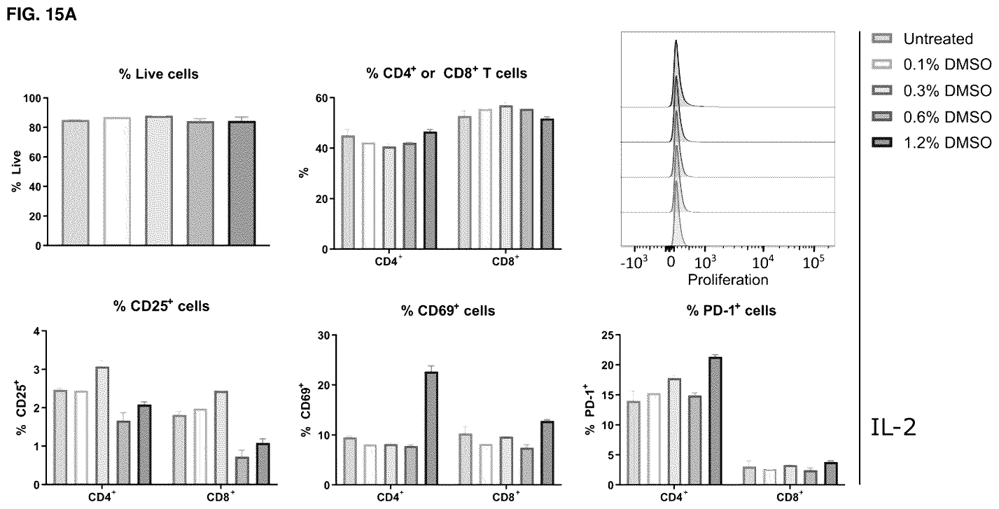

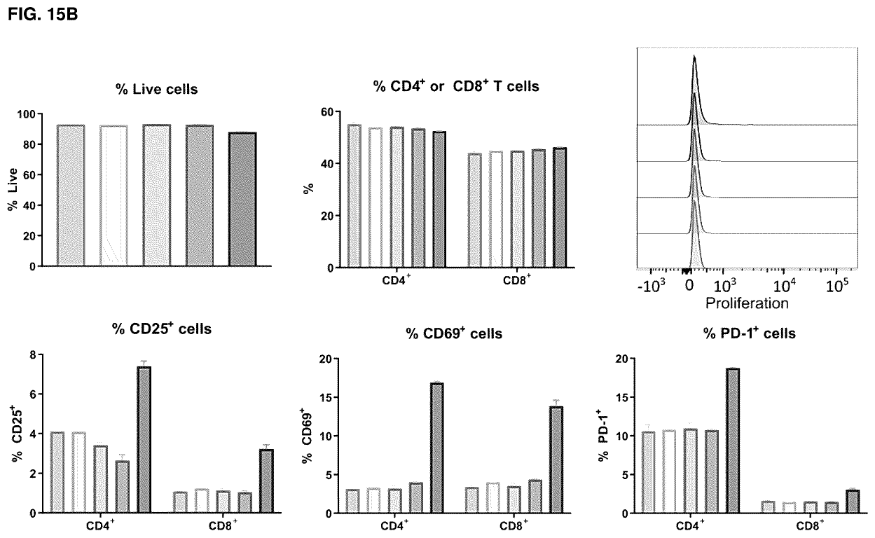

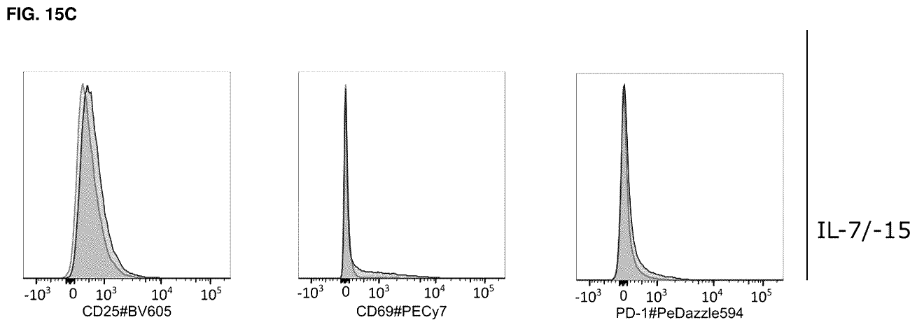

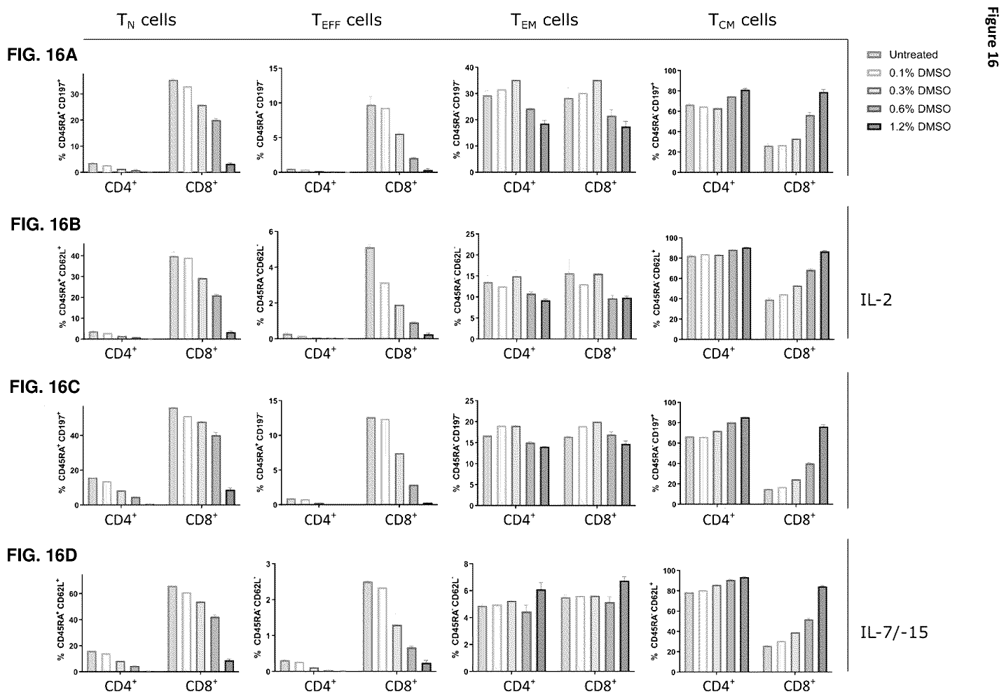

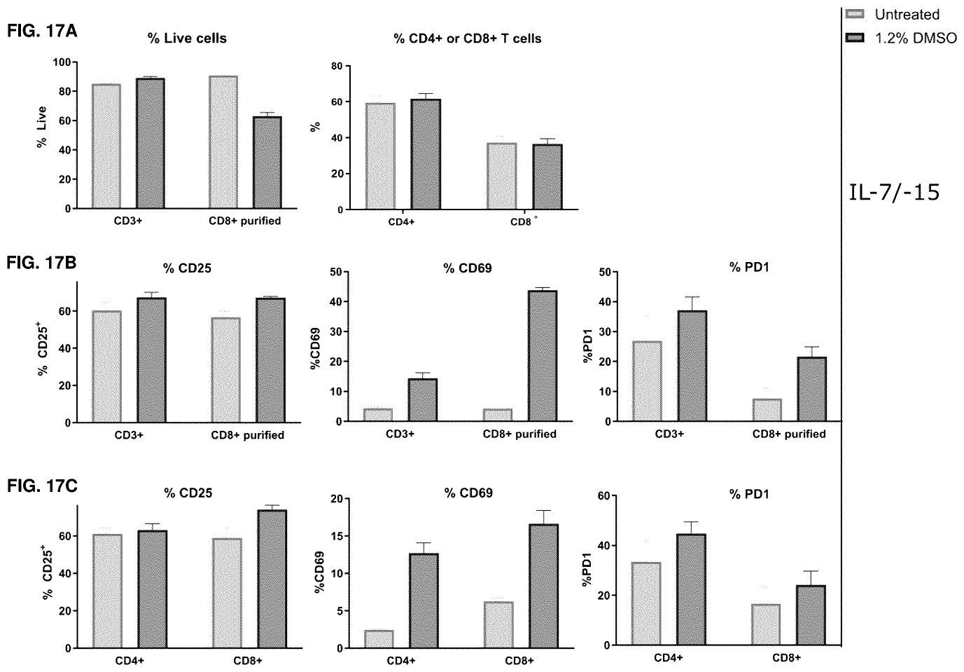

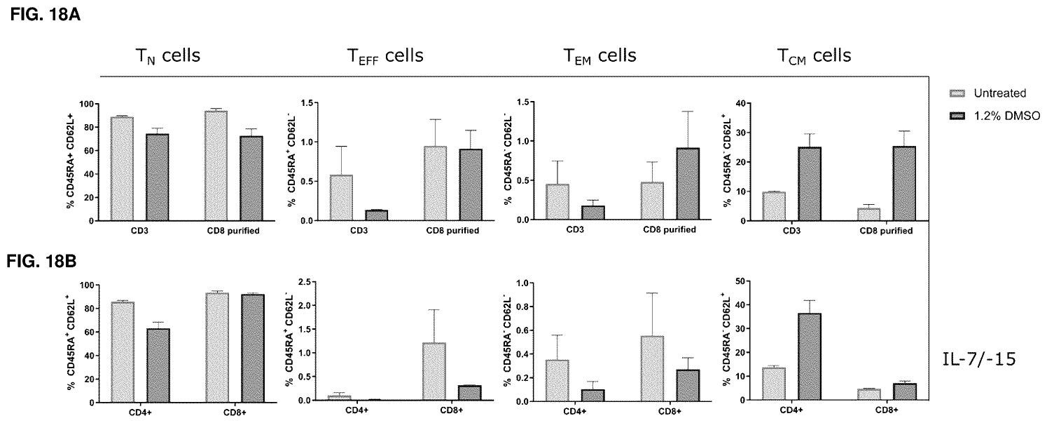

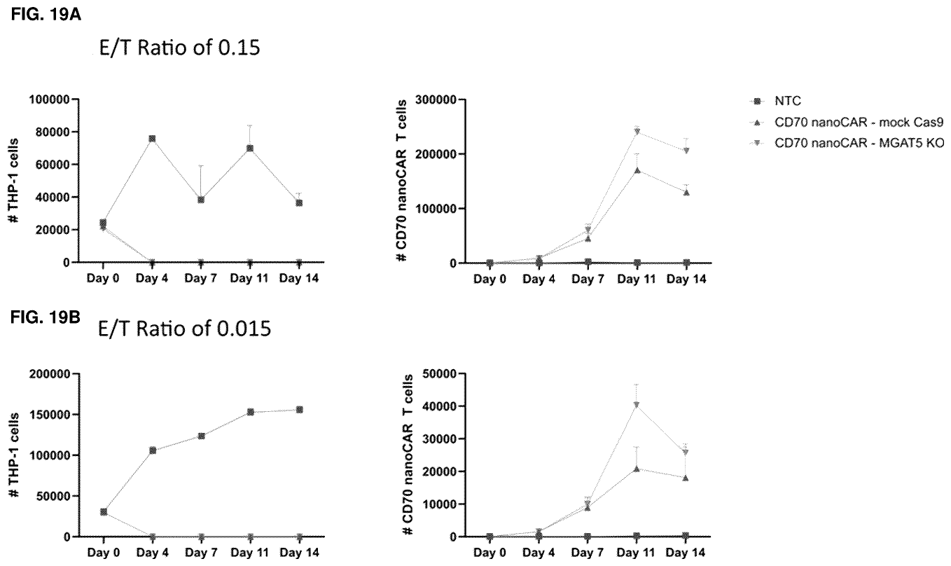

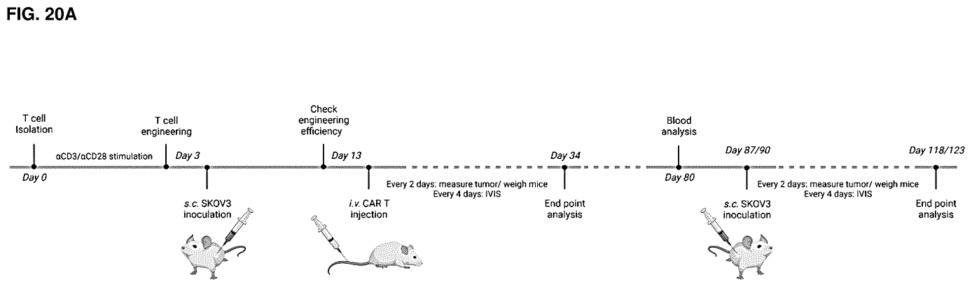

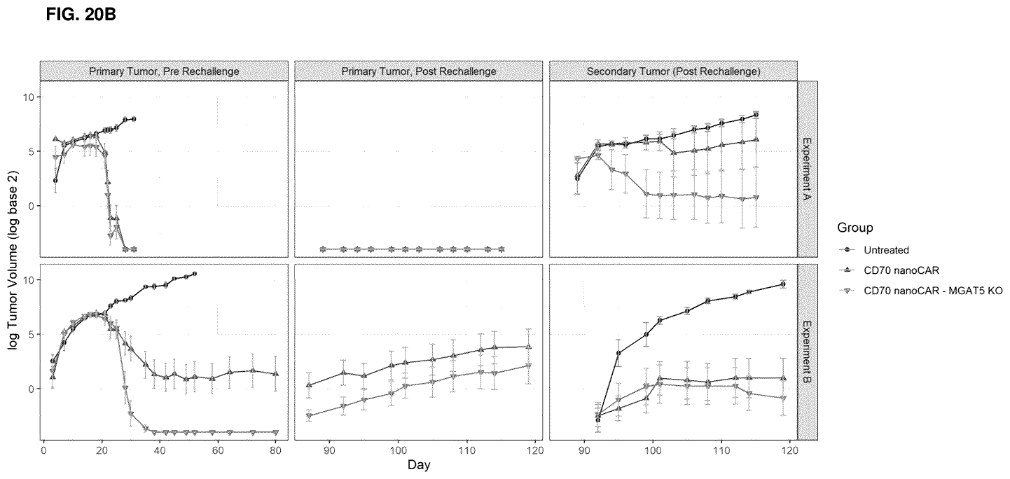

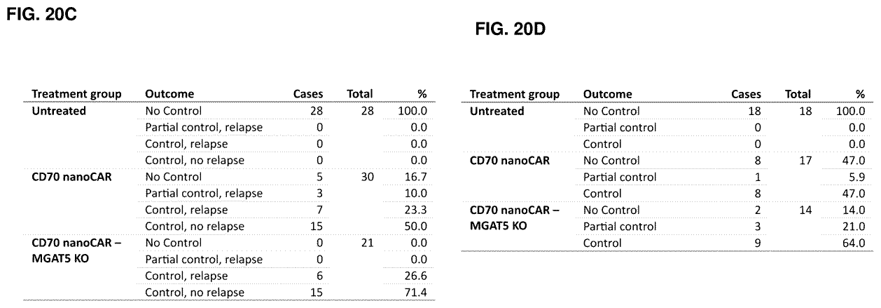

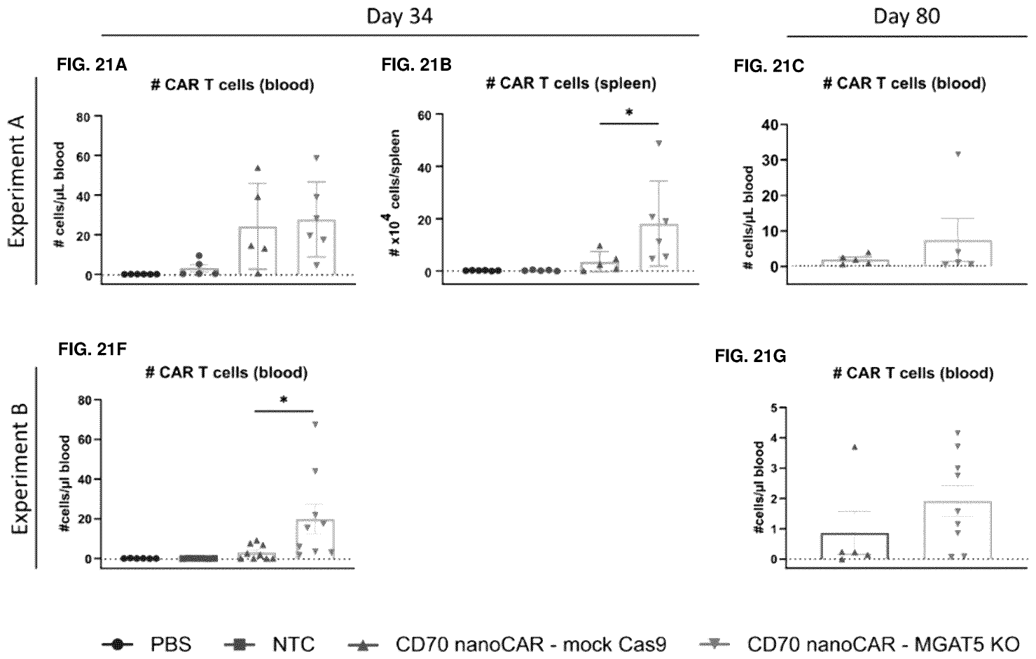

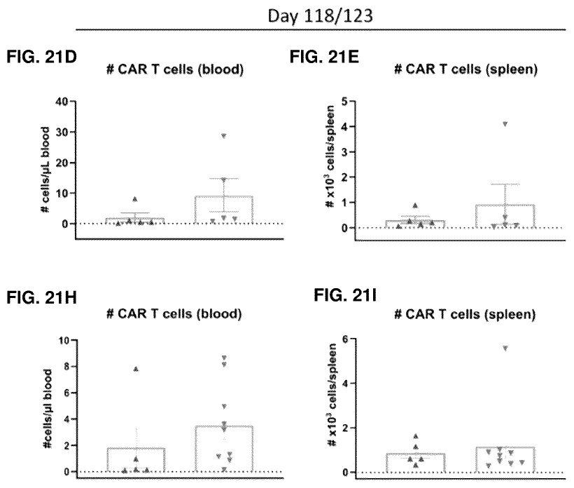

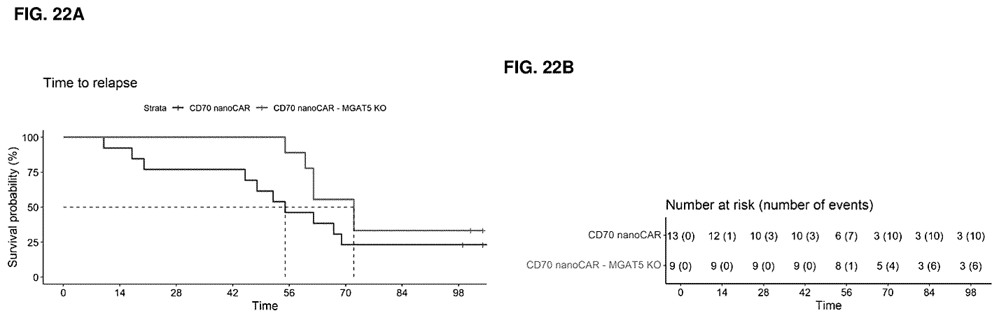

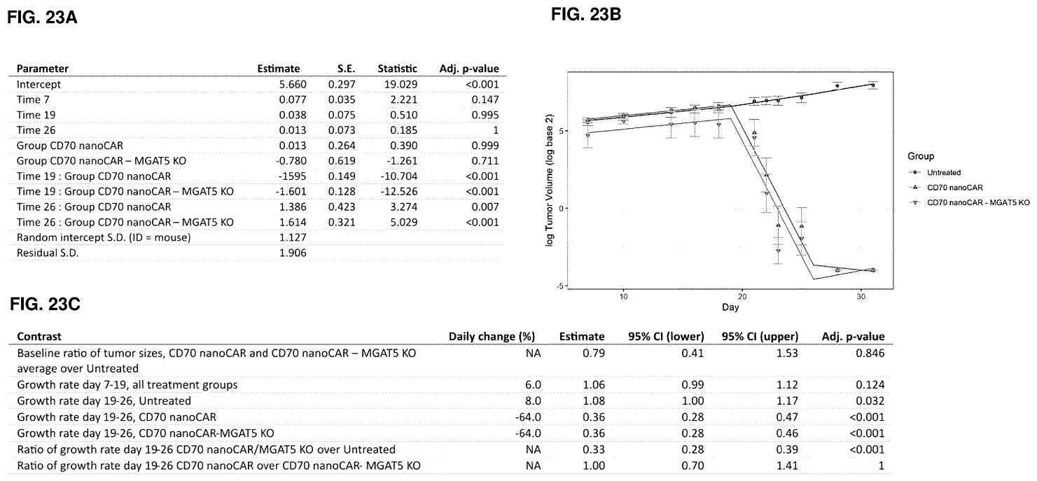

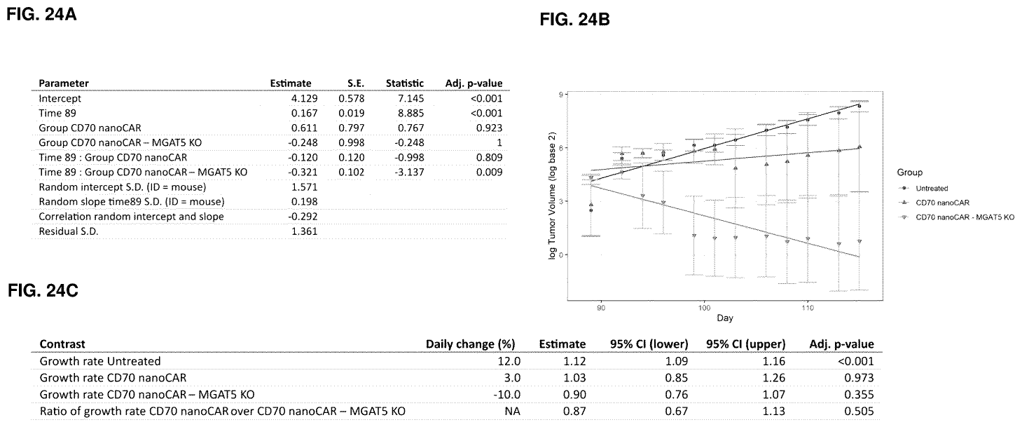

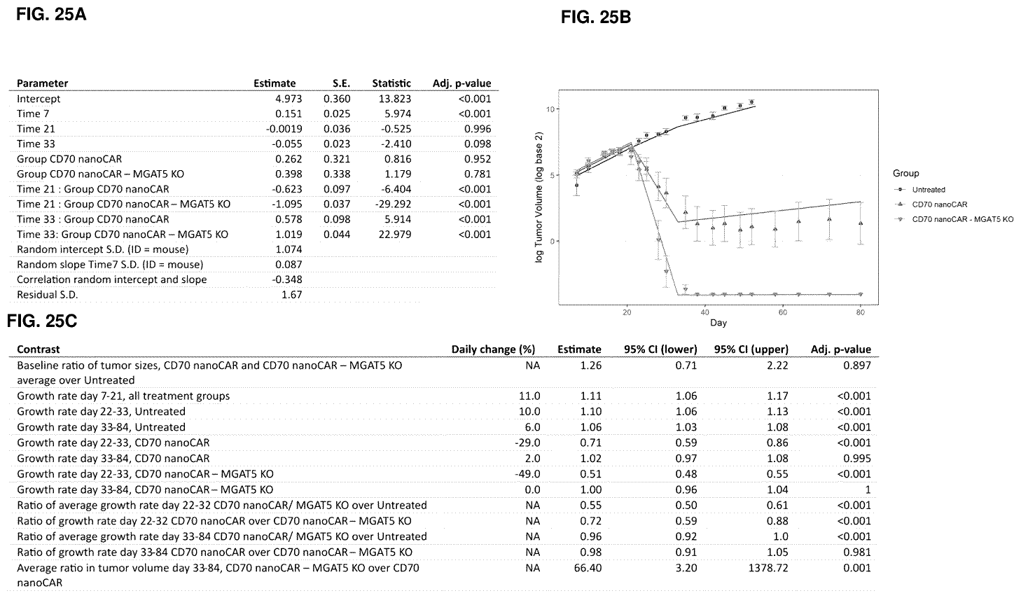

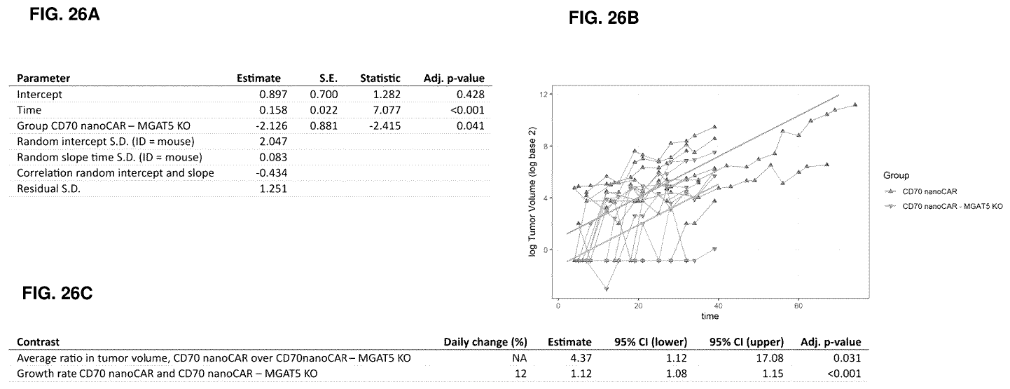

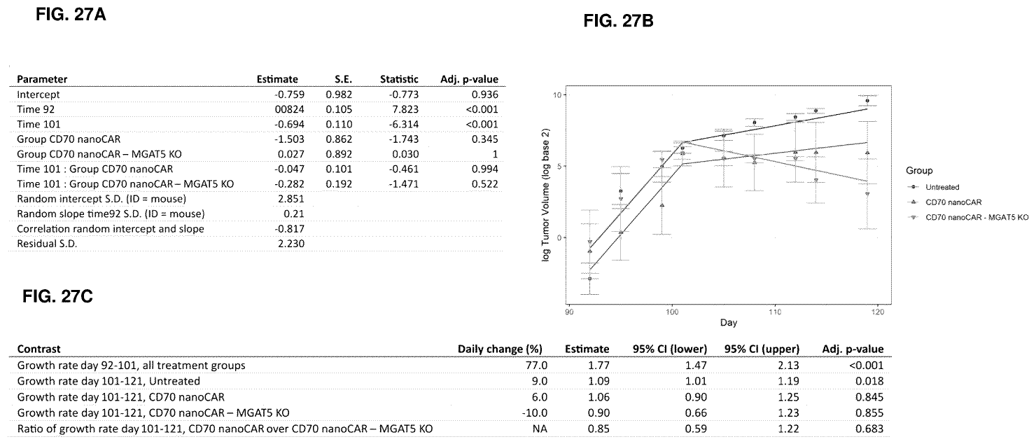

FIG. 3. Schematic representation of the experimental timeline for the study of the in vivo efficacy of MGAT5 KO CD70 nanoCAR-T cells.

FIGS. 9A-9I. Analysis of the in vivo efficacy of MGAT5 KO CD70 nanoCAR-T cells. NSG mice with an established tumor at the right flank were treated with a single dose of 2.5×10⁶ CD70 nanoCAR-T cells that were engineered and cultured in the presence of IL-7 and IL-15 as described. FIG. 9A. Bioluminescence images showing tumor burden in NSG mice at different timepoints post adoptive CAR-T cell transfer (day 13, indicated with orange arrow in panel B). A representative female (left) and male (right) mouse is depicted for each group. FIG. 9B. Tumor burden measured by caliper. Tumor volume is calculated as (tumor length×tumor width²)/2. FIG. 9C. BLI signal obtained through IVIS. Data was analyzed using Living Image Software and represented as photons/second. FIGS. 9D-9I. Flow cytometry-based end-point analysis of the immunophenotype of CAR-T cells in blood or spleen. Each data point represents a single animal. Error bars represent the standard error of the mean (SEM). P-values were calculated by a one-way ANOVA:*, P<0.05 D. The number of human CD3⁺ T cells present in the blood indicated as cells/μL blood (FIG. 9D) and as total number of CD3′ cells in the spleen (FIG. 9G). FIGS. 9E, 9F, 9H and 9I. Immunophenotype of mock Cas9 or MGAT5 KO CD70 nanoCAR-T cells in peripheral blood and spleen. Data is represented as proportion of CD3⁺GFP⁺ cells. T_{N}, naïve T cell; T_{EF}, effector T cell; T_{EM}, effector memory T cell; T_{CM}, central memory T cell.

FIGS. 10A-10D. Evaluation of long-term in vivo (MGAT5 KO) CD70 nanoCAR persistence at day 80. NSG mice that were treated before with (MGAT5 KO) CD70 nanoCAR-T cells remained tumor free. FIG. 10A. Bioluminescence images showing tumor burden in NSG mice at day 85 post adoptive CAR-T cell transfer. A representative female (left) and male (right) mouse is depicted for each group. FIGS. 10B-10D. Persistence of (MGAT5 KO) CD70 nanoCAR-T cells in the blood at day 80. The number of CD3⁺ T cells is indicated as cells/μL blood. Subsets are indicated as percentage of CD3⁺GFP⁺ cells. Each data point represents a single animal. Error bars represent the standard error of the mean (SEM).

FIGS. 11A-11H. Evaluation of long-term in vivo (MGAT5 KO) CD70 nanoCAR persistence. At day 85, a rechallenge experiment was performed by inoculation of 2×10⁶ SKOV-3 cells in the other flank, with MGAT5 KO CD70 nanoCAR, or mock Cas9 CD70 nanoCAR. Additional mice were included as non-treated controls (PBS). FIG. 11A. Tumor burden measured by caliper. Tumor volume is calculated as (tumor length×tumor width²)/2. FIG. 11B. BLI signal obtained through IVIS. Data was analyzed using Living Image Software and represented as photons/second. FIGS. 11C-11H. Flow cytometry-based end-point analysis on day 116 in peripheral blood and spleen. Each data point represents a single animal. Error bars represent the standard error of the mean (SEM) FIGS. 11C & 11F. The number of human CD3⁺ T cells present in the blood indicated as cells/μL blood (FIG. 11C) or the number of cells in the spleen (FIG. 11F). FIGS. 11D, 11E, 11G, 11H, immunophenotype of CAR-T cells in peripheral blood and spleen. Data is represented as proportion of CD3‘GFP’ cells. T_{N}, naïve T cell; T_{EF}, effector T cell; T_{EM}, effector memory T cell; T_{CM}, central memory T cell.

FIGS. 12A and 12B. The effect of increasing DMSO concentrations on human CD8⁺ T cell viability, activation and proliferation. Purified human CD8⁺ T cells were stimulated for 4 days and cultured in the presence of IL-2 (FIG. 12A) or IL-7 and IL-15 (FIG. 12B) and increasing concentrations of DMSO (0.1%-1.2%). Viability, proliferation and expression of the activation markers CD25 and CD69 were analyzed by flow cytometry. Representative histograms are shown for results obtained at day 11 comparing untreated CD8⁺ T cells with CD8⁺ T cells cultured in the presence of 1.2% DMSO. Representative results are shown from independent experiments performed on T cells isolated from three different blood donors.

FIGS. 13A and 13B. The effect of increasing DMSO concentrations on human CD8⁺ T cell exhaustion. Purified human CD8⁺ T cells were stimulated for 4 days and cultured in the presence of IL-2 (FIG. 13A) or IL-7 and IL-15 (FIG. 13B) and increasing concentrations of DMSO (0.1%-1.2%). T cell exhaustion characterized by expression of PD-1, CTLA-4 and TIM-3 was analyzed by flow cytometry. Representative histograms are shown for results obtained at day 11 comparing untreated CD8⁺ T cells with CD8⁺ T cells cultured in the presence of 1.2% DMSO. Representative results are shown from independent experiments performed on T cells isolated from three different blood donors.

FIGS. 14A-14D. The effect of increasing DMSO concentration on human CD8+ T cell differentiation. Purified human CD8+ T cells were stimulated for 4 days and cultured in the presence of IL-2 (FIGS. 14A and 14B) or IL-7 and IL-15 (FIGS. 14C and 14D) and increasing concentrations of DMSO (0.1% -1.2%). T cell differentiation was analyzed by flow cytometry and subsets are described as naïve (CD45RA+CD197+), effector (CD45RA+CD197−), effector memory (CD45RA−CD197−) and central memory (CD45RA−CD197+) T cells in FIGS. 14A and 14C and alternatively as naïve (CD45RA+CD62L+), effector (CD45RA+CD62L−), effector memory (CD45RA−CD62L−) and central memory (CD45RA-CD62L+) T cells in FIGS. 14B and 14D. Results from two independent donors are shown as mean+/−SEM. Results were statistically analyzed as repeated measurements using method of residual maximum likelihood (REML), as implemented in Genstat for Windows 21st edition. Significant results are indicated with * p<0.05, ** p<0.01, *** p<0.001 only when significant.

FIGS. 15A-15C. The effect of increasing DMSO concentrations on human CD3⁺ T cell viability, activation and proliferation. Purified human CD3⁺ T cells were stimulated for 3 days in the presence of IL-12 and cultured in the presence of IL-2 (FIG. 15A) or IL-7 and IL-15 (FIG. 15B) and increasing concentrations of DMSO (0.1% -1.2%). Viability, proliferation and expression of the activation markers CD25 and CD69 and exhaustion marker PD-1 were analyzed by flow cytometry (FIG. 15C). Representative histograms are shown for results obtained for CD3⁺ T cells at day 14.

FIGS. 16A-16D. The effect of increasing DMSO concentration on human CD3⁺ T cell differentiation. Purified human CD3⁺ T cells were stimulated for 3 days in the presence of IL-12 and cultured in the presence of IL-2 (FIGS. 16A and 16B) or IL-7 and IL-15 (FIG. 16C) and increasing concentrations of DMSO (0.1% -1.2%). T cell differentiation was analyzed by flow cytometry and subsets are described as naïve (CD45RA+CD197+), effector (CD45RA+CD197−), effector memory (CD45RA−CD197−) and central memory (CD45RA−CD197+) T cells in panels A and C and alternatively as naïve (CD45RA+CD62L+), effector (CD45RA+CD62L−), effector memory (CD45RA−CD62L−) and central memory (CD45RA−CD62L+) T cells in FIGS. 16B and 16D. Error bars represent 1 SD of two replicate measurements.

FIGS. 17A-17C. The effect of 1.2% DMSO on human CD3+ and CD8+ T cell viability and activation. Purified human CD3+ T cells and CD8+ T cells (from the same donor) were stimulated for 3 days in the presence of IL-12 and cultured in the presence of IL-7 and IL-15 and 1.2% DMSO. Viability (FIG. 17A—left panel) and CD4+/CD8+ T cell subtypes in the CD3+ pool (FIG. 17A—right panel) were analyzed by flow cytometry. Activation markers CD25 and CD69 and exhaustion marker PD-1 were analyzed by flowcytometry, on either the CD3+ pool/CD8+ purified T cells (FIG. 17B) and CD4+ and CD8+ subtypes in the CD3+ pool (FIG. 17C). Representative histograms are shown for results obtained at day 13. Error bars represent standard deviations. The data shown are representative of three independent experiments performed on T cells isolated from three different blood donors.

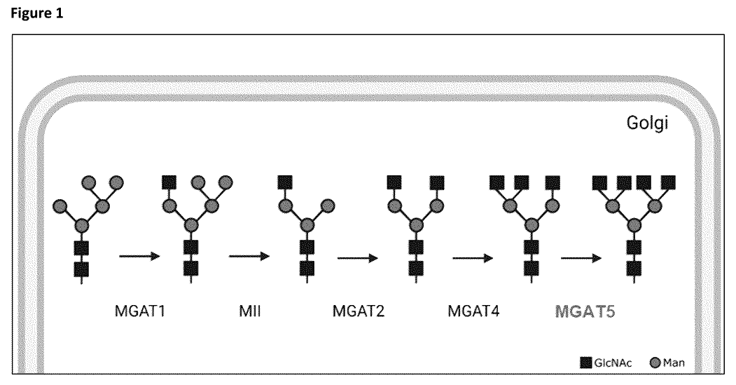

FIGS. 18A and 18B. The effect of increasing DMSO concentration on human CD3 and purified CD8+ T cell differentiation. Purified human CD3+ T cells and human CD8+ T cells from the same donor were stimulated for 3 days in the presence of IL-12 and in the presence of IL-7 and IL-15 (FIGS. 18A and 18B) and 1.2% of DMSO. T cell differentiation was analyzed by flow cytometry and subsets are described as naïve (CD45RA+CD62L+), effector (CD45RA+CD62L−), effector memory (CD45RA−CD62L−) and central memory (CD45RA−CD62L+) T cells. Representative histograms are shown for results obtained at day 13. Error bars represent standard deviations. The data shown are representative of three independent experiments performed on T cells isolated from three different blood donors.

FIGS. 19A-19B. The impact of glyco-engineering in MGAT5 on in vitro CD70 nanoCAR cytotoxic potential. Glyco-engineered CD70 nanoCAR-T cells cultured in the presence of IL-7 and IL-15 were incubated at different effector to target THP-1 cell ratios in duplicate and cell numbers were analyzed over a time period of 14 days. A second challenge with THP-1 cells was added at day 7. Error bars represent the standard error of the mean cell number from data obtained with 3 different T cell donors. FIG. 19A. results for E/T ratio of 0.15 corresponding to the co-culture of 20 000 THP-1 cells with 3 000 glyco-engineered CD70 nanoCAR-T cells. FIG. 19B. Results for E/T ratio of 0.015 corresponding to the co-culture of 20 000 THP-1 cells with 300 CD70 nanoCAR-T cells.

FIGS. 20A-20D. The impact of glyco-engineering on in vivo CD70 nanoCAR functionality. FIG. 20A. Schematic representation of the experimental timeline for the study of the in vivo efficacy of MGAT5 KO CD70 nanoCAR T cells. Timepoints that differ between Experiment A and Experiment B are indicated with a ‘/’. FIG. 20B. Tumor burden measured by caliper. Tumor volume is calculated as (tumor length×tumor width2)/2. Group means are indicated with error bars representing the standard error of the mean (SEM). FIG. 20C. Overview of the response to primary tumor challenge in the different treatment groups. FIG. 20D. Overview of the response to secondary tumor challenge in the different treatment groups.

FIGS. 21A-21I. Flow cytometry-based analysis of CART cells in blood and spleen. Immunophenotype of mock Cas9 or MGAT5 KO CD70 nanoCAR T cells in peripheral blood and spleen at day 34, day 80 and day 118 (Experiment A)/day 123 (Experiment B). Data is represented as proportion of CD3‘GFP’ cells. Each data point represents a single animal. Error bars represent the standard error of the mean (SEM). P-values were calculated by a one-way ANOVA:*, P<0.05. FIGS. 21A, 21C, 21D, 21F, 21G, 21H. The number of CART T cells present in the blood is indicated as cells/μL blood. FIGS. 21B, 21E, 21I. The number of CAR T cells in the spleen is indicated as CD4⁺ or CD8⁺ T cells.

FIGS. 22A and 22B. Time (in days) to relapse of the primary tumor (Experiment B). Time zero was set as the time that the primary tumor was controlled or partially controlled. An event is the time the tumor starts growing again. We take the last day before the tumor has increased in size again or became detectable again as the onset of relapse. FIG. 22A. Kaplan-Meier curve. This plot shows the probability of relapse-free survival in the two groups. The dotted lines indicate median survival times. FIG. 22B. Risk and event table corresponding to the Kaplan-Meier plot. The table shows the number of mice at risk and, between brackets, the cumulative number of relapses in each group and at each time.

FIGS. 23A-23C. Longitudinal analysis of the primary tumor (Experiment A). Using the longitudinal data of the primary tumors in Experiment A, a piecewise linear mixed model with the first timepoint at day 7 and knots at day 19 and 26 and with interactions between the group and the first and second time-segment was fitted, which allows to model the mean traces of each treatment group. FIG. 23A. Summary of the model output, listing all parameter estimates for the model logTumorVol^{˜} Time7+(Time19+Time26))*Group+(1|ID). The table gives parameters and standard errors on the ² log scale together with test statistics and multiple testing adjusted p-values (null hypothesis: parameter equal to zero). FIG. 23B. Plot of the caliper measurements and model fit. The dots are mean ² log tumor volumes with S.E.M. for each group at each day they were measured. The lines are the model-based predictions for the mean ² log tumor volume for each group. FIG. 23C. Inference for different research questions. In this table, the estimates and confidence intervals are transformed back to the original scale so we can interpret them in a straightforward way. E.g. a growth rate of 1.06 means a multiplicative change in tumor volume of 1.06 each day, or a 6% increase each day, compared to the previous day. In this context, the adjusted p-values also relate to a transformed null hypothesis (i.e. estimate equals one). S.E.: Standard Error. CI: Confidence Interval.

FIGS. 24A-24C. Longitudinal analysis of the secondary tumor (Experiment A). A linear mixed model with interactions between the group and time was fitted to the longitudinal data of the secondary tumor in Experiment A, which start at day 89. FIG. 24A. Summary of the model output, listing all parameter estimates for the model logTumorVol^{˜} Time89*Group+(Time89|ID). The table gives parameters and standard errors on the ² log scale together with test statistics and multiple testing adjusted p-values (null hypothesis: parameter equal to zero). FIG. 24B. Plot of the caliper measurements and model fit. The dots are mean ² log tumor volumes with S.E.M. for each group at each day they were measured. The lines are the model-based predictions for the mean ² log tumor volume for each group. FIG. 24C. Inference for different research questions. In this table, the estimates and confidence intervals are transformed back to the original scale so we can interpret them in a straightforward way. S.E.: Standard Error. CI: Confidence Interval.

FIGS. 25A-25C. Longitudinal analysis of the primary tumor (Experiment B). A piecewise linear mixed model with interactions between the group and the second- and third-time segment was fitted to the longitudinal data of the primary tumor in Experiment B. The data start at day 7 and knots are added at day 21 and 33. FIG. 25A. Summary of the model output, listing all parameter estimates for the model logTumorVol^{˜} Time7+(Time21+Time33)*Group+(Time7|ID). The table gives parameters and standard errors on the ² log scale together with test statistics and multiple testing adjusted p-values (null hypothesis: parameter equal to zero). FIG. 25B. Plot of the caliper measurements and model fit. The dots are mean ² log tumor volumes with S.E.M. for each group at each day they were measured. The lines are the model-based predictions for the mean ² log tumor volume for each group. FIG. 25C. Inference for different research questions. In this table, the estimates and confidence intervals are transformed back to the original scale so we can interpret them in a straightforward way. S.E.: Standard Error. CI: Confidence Interval.

FIGS. 26A-26C. Longitudinal analysis of the relapse of the primary tumor (Experiment B). A linear mixed model was fitted to the longitudinal data of the relapsing mice in Experiment B. Only mice in the treated groups had cleared the tumors fully or partially, so the analysis is naturally restricted to the two CAR groups. To enable this analysis, we also had to change the timescale for each individual mouse such that the first day of the relapse became day 0. Had we not done this, the analysis would be moot since, on average, the CD70 nanoCAR group had relapses earlier than the CD70 nanoCAR-MGAT5 KO group. This would almost automatically result in larger tumors in the CD70 nanoCAR group compared to the CD70 nanoCAR-MGAT5 KO group. FIG. 26A. Summary of the model output, listing all parameter estimates for the model logTumorVol^{˜} Time+Group+(Time|ID). The table gives parameters and standard errors on the ² log scale together with test statistics and multiple testing adjusted p-values (null hypothesis: parameter equal to zero). FIG. 26B. Plot of the caliper measurements and model fit. The dots are individual ² log tumor volumes. Dots connected by a line are measurements from the same mouse (note that due to the time translation, mean values per day are not informative, since not all measurements were made on the same day on the new timescale). The two straight lines are the model-based predictions for the mean ² log tumor volume for each group. FIG. 26C. Inference for different research questions. In this table, the estimates and confidence intervals are transformed back to the original scale so we can interpret them in a straightforward way. Note that the interaction between Time and Group was not significant here so the growth rate is the same in each group but the tumors are smaller on average in the MGAT KO group. S.E.: Standard Error. CI: Confidence Interval. FIGS. 27A-27C. Longitudinal analysis of the secondary tumor (Experiment B). A piecewise linear mixed model with interactions between the group and the second time segment was fitted to the longitudinal data of the secondary tumor in Experiment B. The data start at day 92 and a knot is added at day 101. FIG. 27A. Summary of the model output, listing all parameter estimates for the model logTumorVol^{˜} Time92+Time101*Group+(Time92|ID). The table gives parameters and standard errors on the ² log scale together with test statistics and multiple testing adjusted p-values (null hypothesis: parameter equal to zero). FIG. 27B. Plot of the caliper measurements and model fit. The dots are mean ² log tumor volumes with S.E.M. for each group at each day they were measured. The lines are the model-based predictions for the mean ² log tumor volume for each group. FIG. 27C. Inference for different research questions. In this table, the estimates and confidence intervals are transformed back to the original scale so we can interpret them in a straightforward way. S.E.: Standard Error. CI: Confidence Interval.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology). More specifically, in the field of immunology, reference is made to Janeway et a., Immunobiology: the immune system in health and diseases, 4th ed., Elsevier Science Ltd/Garland Publishing, 1999. The terms specific to the field of CAR-T immunotherapy as known to the skilled person are referred to as in for instance Feins et al.: An introduction to chimeric antigen receptor (CAR) T-cell immunotherapy for human cancer; Am J Hematol. 2019; 94:S3-S9.; Fesnak et al. Engineered T Cells: The Promise and Challenges of Cancer Immunotherapy, Nat Rev Cancer. 2016; 16(9): 566-581; Fesnak, et al. CAR-T cell therapies from the transfusion medicine perspective. Transfus Med Rev (2016) 30, 139-145.; Wang & Rivière, Clinical manufacturing of CAR-T cells: foundation of a promising therapy; Oncolytics (2016) 3, 16015; unless specifically defined herein otherwise.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence, post-translational modifications and/or functional properties (i.e., altered characteristics) when compared to the wild-type gene or gene product. It is noted that naturally occurring mutants can be isolated; these are identified by the fact that they have altered characteristics when compared to the wild-type gene or gene product. A knock-out refers to a modified or mutant or deleted gene as to provide for non-functional gene product and/or function. It is noted that naturally occurring mutants or variants may be isolated; these are identified by the fact that they have altered characteristics when compared to the wild-type gene or gene product, and a different sequence as compared to the reference gene or protein.

The term “vector”, “vector construct,” “expression vector,” or “gene transfer vector,” as used herein, is intended to refer to a nucleic acid molecule capable of transporting another nucleic acid molecule to which it has been linked. More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the terms “determining,” “measuring,” “assessing,”, “identifying”, “screening”, and “assaying” are used interchangeably and include both quantitative and qualitative determinations.

The terms “subject”, “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, fish, reptiles, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present.

The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

The terms “prevents”, “inhibits”, “blocks” or “reduces” a certain state of condition are used interchangeably and mean the observed degree of the state of condition is significantly lower in comparison to state or condition observed when the CAR-T cell composition of the invention is present.

The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein.

This invention also relates to “pharmaceutical compositions” comprising the (CAR-) T cell or (CAR-) T cell composition of the invention, and optionally a pharmaceutically acceptable carrier or diluent. These pharmaceutical compositions can be utilized to achieve the desired pharmacological effect by administration to a patient in need thereof. A “pharmaceutically or therapeutically effective amount” of compound or cellular population or composition is preferably that amount which produces a result or exerts an influence on the particular condition being treated. The CAR-T cell composition or the pharmaceutical composition comprising said CAR-T cells as described herein may also function as a “therapeutically active agent” which is used to refer to any molecule that has or may have a therapeutic effect (i.e. curative or stabilizing effect) in the context of treatment of a disease (as described further herein), preferably in the context of immunotherapy in treatment of cancer. Preferably, a therapeutically active agent is a disease-modifying agent, and/or an agent with a curative effect on the disease. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and cryopreservatives. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), van der Walle, et al. “Formulation Considerations for Autologous T Cell Drug Products”; Pharmaceutics 2021, 13, 1317; and Nema, S. et al. (“Excipients and Their Use in Injectable Products” PDA Journal of Pharmaceutical Science & Technology 1997, 51 (4), 166-171). The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

The term “administration” to a subject as used herein refers to any route of introducing or delivering an agent, such as a CAR-T cell composition or pharmaceutical composition to a subject. Administration can be performed using any suitable route, including oral, topical, intravenous, subcutaneous, transdermal, intramuscular, intra-articular, parenteral, intra-arteriolar, intradermal, intraventricular, intracranial, intraperitoneal, intralesional, intranasal, rectal, vaginal, by inhalation, by implanted reservoir, parenteral (e.g., subcutaneous, intravenous, intramuscular, intra-articular, intrasynovial, intrasternal, intrathecal, intraperitoneal, intrahepatic, intralesional and intracranial injection or infusion techniques), and the like.

### DETAILED DESCRIPTION

The first aspect of the present invention relates to a Chimeric antigen receptor (CAR) T cell, more specifically a human CAR-T cell, which is genetically modified to contain a mutation in the Mgat5 gene. Said MGAT5 mutant or knock-out CAR-T cell is thus deficient in N-glycan branching leading to tetra-antennary N-glycans, providing for human CAR-T cell which are devoid of said glycans at their cell surface. The chimeric antigen receptor or ‘CARs’ refers to modular synthetic receptors that consist of four main components: (1) an extracellular target antigen binding domain, (2) a hinge region, (3) a transmembrane domain, and (4) at least one intracellular signaling domain, driving CAR-T cell effector functions. Chimeric Antigen Receptors are construed with domains derived from different origins. For durable T cell activation, co-stimulatory signaling is also required (used in 2^{nd} and further generation CARs). CAR-T cells provide for effective killing of target tumor cells, and optimally also persist and last as sustainable immune system gatekeepers.

The surface glycan structures present on T cells can be engineered at different levels. Poly-N-acetyllactosamine (PolyLacNAc) comprises repeated Galactose (Gal)-β1-4-Nacetyl-glucosamine (GlcNAc) disaccharides, called N-acetyllactosamine (LacNAc). The biosynthesis of poly-LacNAc is accomplished by the enzymatic action of β-1,3 N-acetylglucosaminyltransferases which catalyze the addition of GlcNAc to N-glycan Galactose (Gal) termini. Poly-LacNAc structures are built onto the β1,6-GlcNAc initiated branch introduced by N-acetylglucosaminyltransferase V (MGAT5) (FIG. 1). β-1,6-N-acetylglucosaminyltransferase-V (MGAT5) is the enzyme responsible for the initiation of GlcNAc-β-(1,6)-branching on N-glycans, which leads to an increase in LacNAc modifications, the ligand of Galectins.

The human CAR-T cell as described herein is genetically modified to provide for a mutated Mgat5 gene, thereby affecting surface N-glycans characterized in that the surface of the T cells lacks tetra-antennary N-glycans at its cell surface. Genetic modification of (human) T cells as used herein refers to the process of modifying a cell or cell population, such as T cell, or CAR-T cell, with genetic material such as a nucleic acid molecule or vector, plasmid, or a ribonucleoprotein, that has been designed or synthesized using molecular technology, and transformed or transduced in said cell, in a stable or transient manner. As a result, nucleotide changes occur as substitutions, deletion or insertions of nucleotides, resulting in genes being deleted, mutated, inserted or knocked-down, or knocked-out using genetic modification technologies as known in the art, such as for instance transformation using plasmids for introducing antisense oligo's or RNA, RNAi, or alternative transgene delivery in a DNA-dependent manner (e.g., lentiviral and retroviral transduction). Furthermore, DNA-free genome editing using an endonuclease ribonucleoprotein complex (such as, for example, Cas9/RNP) may also be used herein. The endonuclease/RNP (e.g., Cas9/RNP) consists of three components, a recombinant endonuclease protein (e.g., Cas9 endonuclease) that is complexed with a CRISPR locus. The endonuclease complexed to a CRISPR locus may be referred to as a CRISPR/Cas guide RNA. CRISPR loci comprise synthetic single guide RNAs (grnas) comprising RNA that can hybridize to complex complementary repeat RNAs (crrnas) and trans complementary repeat RNAs (tracrrnas) of a target sequence. Thus, the CRISPR/Cas guide RNA hybridizes to a target sequence in the genomic DNA of the cell. Compared to exogenous DNA-dependent methods, these Cas9/RNPs are able to cleave genomic targets with higher efficiency because they are delivered in the form of a functional complex.

Thus, in one embodiment disclosed herein the human CAR-T cell as described herein is genetically engineered specifically in its Mgat5 gene, resulting in the absence of functional MGAT5, wherein said engineering is preferably obtained using a guide RNA (grna) specific for said Mgat5 target DNA sequence in the T cells, and transducing (e.g., introducing by electroporation) into the T cell a Ribonucleoprotein (RNP) complex comprising a CRISPR/Cas endonuclease (Cas9) complexed to a corresponding CRISPR/Cas guide RNA that hybridizes to the Mgat5 DNA sequence within the genomic DNA of the T cell.

Genetic modification performed by transduction is frequently used to modify T cells to include CARs, and “transduction” refers to methods for transferring genetic material, such as, for example, DNA or RNA, into a cell via a vector. Conventional techniques use viral vectors, electroporation, transposon transfection, and chemicals to increase cell permeability. DNA can be transferred via a virus or via a viral vector. As described herein, methods of modifying immune CD4+ and/or CD8+ T cells are provided. In order to achieve high levels of expression of therapeutic genes and/or to increase the number of chimeric antigen receptors on the cell surface, for example, T cells can be transduced with genetic material encoding a chimeric antigen receptor. Reliable CAR gene delivery can be obtained using electroporation or viral vectors from murine-derived retroviruses or lentiviruses (Zhao, et al. Cancer Res 70, 9053-9061 (2010); Singh, et al. PLoS One 8, e64138 (2013). T cells can be genetically modified using a virus. Viruses commonly used for gene therapy are adenovirus, adeno-associated virus (AAV), retroviruses, and lentiviruses. Various transduction techniques have been developed that use recombinant infectious viral particles to deliver nucleic acid encoding a chimeric antigen receptor. Viral vectors used for transduction may include viral vectors derived from monkey virus 40, adenoviruses, adeno-associated virus (AAV), lentiviral vectors and retroviruses. Primary T lymphocytes have been successfully transduced by electroporation and by retroviral or lentiviral infection. Thus, vectors based on retroviruses and lentiviruses can provide a highly efficient way of transferring into T cells. Moreover, the insertion of a retrovirus or lentivirus occurs in a controlled manner and leads to the stable insertion of one or more copies of new genetic information into the cell. Next generation CAR-T cells require multiple genetic modifications, which may be included through gene editing. ZFNs, TALENs, meganuclease-TALENs, and CRISPR RNA-guided nucleases specifically may be applied as editing tool and deliver transgenes or modify DNA sequences based on amino acid sequence of the targeting region.

The human CAR-T cell as described herein, comprising a mutated Mgat5 gene, and/or expressing a CAR, may be obtained using any technology available to the skilled person for transgene delivery and genetic modification or gene editing, suitable for mammalian cells, in particular T cells (see for instance delivery methods described in Mohanty et al. (2010, Oncology reports, 42: 2183)).

### Immunotherapy Using Glyco-Engineered CAR-T Cells

A second aspect relates the MGAT5 mutated human CAR-T cells as described herein, or use as a medicine, more specifically for use in treatment of cancer. One embodiment thus relates to a method to treat a subject comprising the steps of administering the MGAT5 mutated human CAR-T cell composition as described herein.

CAR-T cell therapy has emerged as a novel therapeutic T cell engineering practice, wherein the cells are produced by in vitro engineering of T cells derived from patient blood to express artificial receptors targeted to a specific tumor antigen, allowing to directly identify the tumor antigen without the involvement of the major histocompatibility complex. T cells engineered to express CARs induce high rates of clinical responses in patients with relapsed/refractory hematologic cancer, with a reduction in remission rates of up to 80%, particularly for acute lymphoblastic leukemia (ALL) and non-Hodgkin lymphomas, such as large B cell lymphoma. Moreover, increasing involvement of several auxiliary techniques, such as bispecific CAR, Tan-CAR, inhibitory-CAR, combined antigens, the clustered regularly interspaced short palindromic repeats gene-editing tool and nanoparticle delivery, may substantially improve its overall anti-cancer effects. CAR-Therapy has the potential to offer a rapid and safer treatment regime to treat non-solid and solid tumors (Mohanty et al. 2010, Oncology reports, 42: 2183).

So, different types of cancer that may be treated using the glyco-engineered MGAT5 mutated human CAR-T cells or compositions described herein, different in the nature and specificity of their CARs as applicable for the specific antigens for said patient(s), including the oncological disorders such as for instance, but not limited to, Leukemia, B-cell lymphoblastic leukemia (BALL, B-CLL), Acute myeloid leukemia (AML), Lymphoma, Non-Hodgkins Lymphoma, B-Non Hodgkins Lymphoma, B-NHL/CLL, Mantel cell leukemia/B-NHL, B-cell malignancies, Hepatocellular carcinoma, Neuroblastoma, Prostate cancer, Breast cancer, Colorectal cancer, Lungs cancer, Lung squamous cell carcinoma, Osteosarcoma, Glioblastom, Malignant pleural mesothelioma, Pancreatic cancer, Ovarian carcinoma, Renal cancer, stomach cancer, and Esophageal cancer.

In a further embodiment, the human MGAT5 mutated CAR-T cell or composition may be used in treatment of cancer wherein said treatment prevents, inhibits, blocks, cures, or at least significantly controls or reduces primary and/or secondary or recurrent tumor growth and/or primary and/or secondary or recurrent tumor burden in a subject or patient. Moreover, the human MGAT5 mutated CAR-T cell or composition may be used in treatment of cancer wherein said treatment prevents, inhibits, blocks, or at least significantly reduces relapse of tumor formation or cancer in a subject. Indeed, after primary challenge, treatment with human MGAT5 mutated CAR-T cells show an improved effect on primary tumor growth and tumor burden as compared to non-glyco-engineered CAR-T cell treatment. Moreover, and even more pronounced, after treatment of a primary tumor with the human MGAT5 mutated CAR-T cells, an additional subsequent tumor challenge in mice revealed that the administration of the glyco-engineered CAR-T cells described herein as treatment of the primary tumor, provided for a sustained memory, resulting in a significantly lower risk to develop a secondary tumor in said mice, as compared to the control treatment. The term ‘decreased’, ‘reduced’, ‘prevent’ or ‘inhibit’ used herein refers to a reduction of at least 10%, 15%, 20%, 25%, 30%, 40%, 50%, or more than 50% of its effect as compared to a control without the CAR-T cells that contained wild-type MGAT5, or with a negative vehicle control. The term ‘block’ of relapse or tumor growth or burden refers herein to a reduction of secondary tumor development in said subject to a non-detectable level as compared to a control CAR-T treatment with wild type glycans on the cell surface, or another vehicle or negative control. A ‘negative control’ or ‘irrelevant control’ or ‘control’ or ‘vehicle control’ as referred to herein is meant a T cell population of similar nature (e.g. a CAR-T cell different in having wild-type MGAT5 expression), or a buffer control (e.g. PBS) as negative control treatment, having no effect at all, or a vehicle control (e.g. T cells containing an ‘empty’ CAR-Transgene without the target specific effect). A ‘control’ may be one type of molecule or a pool of molecules known to have no effect on the tumor.

The observation the glyco-engineered CAR-T cells as described herein, mutated in Mgat5, have an enhanced impact on the inhibition of primary as well as secondary tumor growth and tumor burden, also indicates these cell compositions provide for an improved response to immunotherapy, which may be caused by the presence of a higher population of memory phenotype cells (see below), or a different mechanism and impact of the altered glycan composition in the TME. Indeed, a growing number of studies suggest that baseline tumor burden, or tumor size, predicts response to immunotherapy, and total tumor burden may act as an important negative correlate of response. Mechanistically, large tumors exert greater local and systemic changes to the immune system, and harbor more immunosuppressive cells and molecules that dampen antitumor activity. Many of the alterations locally and systemically reflect a more immunosuppressive tumor microenvironment (Kim et al. (2021) Front. Immunol. 11:629722).

CAR-T immunotherapy treatment involves “adoptive immunotherapy” which is defined as the infusion of immunocompetent cells for the treatment of cancer. Adoptive cell transfer (ACT) is a robust form of immunotherapy for treatment of established tumors. A certain level of CAR-T-cell expansion and persistence is necessary to induce meaningful tumor regressions, but the predictive indicators and mechanism(s) associated with remarkable proliferation, persistence and favorable clinical responses are largely unknown, which complicates the prediction of patient-responsiveness to CAR-T immunotherapy. To enhance the potency and sustain the function of CAR-T cells in vivo after ACT and to develop mechanism-based approaches to increase the resistance of CAR-T cells to immune-senescence and exhaustion, improved production methods are required as to improve final quality of CAR-T cells for ACT and for maximization of therapeutic effects.

Production of CAR-T Cells with Improved Memory Phenotype

CAR-T cell expansion and persistence seem to be key efficacy determinants in cancer patients, which are both features typical of early-memory T cells, which can be enriched for using specific manufacturing procedures. Moreover, the fraction of central memory T cells the CAR-T cell composition has been shown to positively correlate with in vivo expansion, which is crucial for achieving long-term remissions (see for instance Arcangeli et al. 2020. Front. Immunol. 11:1217; Arcangeli et al. 2022. J Clin Invest. 15; 132 (12):e150807). Depending on the cancer type however, the primary sample for production of the T cells may proportionally contain more exhausted and less of such central memory T cells. Arcangeli et al. (2020. Front. Immunol. 11:1217) also indicate that next-generation manufacturing protocols providing for T_{CM}-enriched CAR-T cell compositions may be qualitatively equivalent to the ones generated from healthy donors.

So increasing evidence suggests that beneficial properties required for persistence and long lasting effects can be enhanced by enriching for early memory CAR-T cell subsets, e.g., stem cell memory (T_{SCM}) and central memory (T_{CM}) T cells. In addition, solid tumor treatments require CAR-T cell composition to traffic to the tumor sites, recognize tumor cells and expand in an extremely immunosuppressive environment. Therefore, CAR-T cell production should be improved to cope with all such challenges, and take into account that depending on tumor context and disease-specific factors, different levels of complexity have to be dealt with. So improvement of the manufacturing protocols represents one of the major goals of current research in the CAR-T field.

The production typically starts with autologous cells, isolated from a patient, and ends with an expanded, modified and viable immunotherapy formulated composition to be re-infused to patients. Ex vivo modification, activation or stimulation, and expansion require sophisticated equipment and expertise, and manufacturing is performed under Good Manufacturing Practices (cGMP) to guarantee a robust quality, maintain product stability of the formulated product used for ACT in patients.

So this application further discloses a method for enhancing memory phenotype in a T-cell, wherein said method comprises the step of:
(a) Isolating T cells from a primary sample,
(b) incubating the T cells of step a) under stimulating conditions, thereby generating a stimulated T cell composition, wherein the T cell composition comprises at least a concentration of 1×10⁶ cells/mL,
(c) introducing a mutation in T cells in the Mgat5 gene, as to obtain a mutated MGAT5 T cell composition, and
(d) cultivating the stimulated T cell composition for expansion of the mutated MGAT5 T cell composition.

A specific embodiment relates to said method wherein the T cells are human T cells. A further specific embodiment relates to said method wherein the memory phenotype of the resulting T cell composition comprises at least 10% more T_{CM} cells as compared to the human T cell composition that is not mutated in the Mgat5 gene, or at least 20% more, at least 30% more or at least 40% more memory T cells as compared to human T cell composition that is not mutated in the Mgat5 gene.

Another specific embodiment relates to said method comprising a further step, performed after step b) and before, simultaneously or after step c), said step comprising:
engineering the stimulated T cell composition by contacting the cells with an agent comprising a polynucleotide encoding the recombinant receptor, as to provide for a mutated MGAT5 CAR-T cell composition.

Said contacting may be performed using the currently known technologies for introducing the CAR into the cells, such as electroporation, using viral vectors, transposon transfection, or nucleofection, among others, as known to the skilled artisan.

The primary sample for step a) of the method as described herein is obtained from a patient or a healthy donor, which often is collected using leukapheresis, an efficient centrifugation-based method for collecting large numbers of mononuclear cells (MNC), including T cells. The primary sample may thus be a blood sample, a buffy coat sample (total nuclear cells), a MNC sample, or a population of cells comprising T cells, preferably human T cells. T cells are isolated or enriched from said primary sample via a variety of methods known to the skilled artisan, such as density gradients to remove non-MNC contaminants such as granulocytes and red blood cells, separations based on cell size and density to isolate lymphocytes from monocyte fractions, or antibody-bead conjugates to obtain pure T cell subsets with high specificity via magnetic separation. Once T cells are isolated, the stimulation of the T cell population is required as to activate the intracellular signaling domains of the TCR complex and/or of the costimulatory molecules. Said stimulation may thus be performed by incubation of the cells in stimulating conditions by the presence of a stimulatory reagent which is capable of activating one or more intracellular signaling domains of one or more components of a T-cell receptor (TCR) complex and/or one or more intracellular signaling domains of one or more costimulatory molecules, such as the stimulatory reagents known in the art, and as provided herein. Since optimal T cell activation results from cognate antigen recognition, co-stimulation, and also cytokine support, so ‘stimulatory reagents’ are defined herein as to contain a primary agent that specifically binds to a member of a TCR complex, and optionally contain a secondary agent that specifically binds to a T cell costimulatory molecule, and optionally, one or more cytokines to further support activation. In certain embodiments of any of the provided methods, the primary and/or secondary agents of the stimulatory agent or conditions comprise a target-specific antibody, and cytokine molecules such as IL-2 or IL-7 and IL-15.

Specifically to provide for stimulating conditions suitable for CAR-T cell production for use in solid tumors, see also the specific stimulatory agents as disclosed in Harrison et al. (2021, Immunotherapy Advances, Vol. 1, No. 1, 1-9).

In some embodiments of any of the provided methods, the primary agent and/or secondary agent are present on the surface of a solid support. In particular embodiments of any of the provided methods, the solid support is or comprises a bead, which may be an inert bead, such as a magnetic or superparamagnetic bead. Said bead may be coated with specific antibodies for providing stimulating conditions. The stimulating conditions as exemplified herein provide for the non-limiting use of for instance, Immunocult™ or Dynabeads coated with target-specific antibodies, and as provided by the manufacturer of said stimulating agents.

As described above, the improvement of production methods for (CAR) T cells for engrafting and ACT in a patient, and provide for a persistent effect, requires that the formulated (CAR) T cell composition has a higher fraction of memory phenotype cells. The present invention further provides for a method to increase said memory phenotype by addition of Dimethyl sulfoxide (DMSO) during the activation and expansion of the T cells ex vivo. DMSO is known as an amphipathic organic solvent and is widely used in biological applications. It is routinely applied as a cryoprotectant for long-term cell freezing as well as to dissolve peptides or drugs for immune cell functional assays. When used in low amounts, DMSO has been shown herein to positively affect in vitro culturing of T cells in the presence of different cytokine combinations in terms of survival, proliferation, activation, exhaustion and differentiation. Most importantly, a method is provided herein with addition of a certain range of DMSO in the medium to skew the differentiation of the expanding T cell composition towards a higher T_{CM} fraction, which may be a major breakthrough for the field of adoptive immune therapy for cancer, where it has been established that T cells with a memory phenotype exert superior anti-cancer immune responses.

So another embodiment provides for a method to produce a T cell composition with a predominant memory phenotype comprising the steps of:
(a) Isolating T cells from a primary sample,
(b) incubating the input T cell composition of step a) under stimulating conditions comprising 0.3-1.2% (v/v) DMSO, thereby generating a stimulated T cell composition, and wherein the input T cell composition comprises at least a concentration of 1×10⁶ cells/mL,
(c) cultivating the stimulated T cell composition in a medium comprising 0.3-1.2% (v/v) DMSO.

Another embodiment provides for said method to produce a CAR-T cell composition with a predominant memory phenotype, further comprising the following step after step b):
(b-bis) engineering the stimulated T cell composition by contacting the cells with an agent comprising a polynucleotide encoding the recombinant receptor.

In a further specific embodiment, said method is provided to produce MGAT5 mutated T cells with a predominant memory phenotype, comprising a further step b-tris) after step b), and/or before, after or simultaneously with step b-bis):
introducing a mutation in T cells in the MGAT5 gene, optionally using CRISPR/Cas engineering.

Said method as described herein for production of a (CAR-)T cell composition with predominant memory phenotype, defines the ‘predominant’ memory phenotype as said T cell composition comprising at least 10% of its cells showing a central memory T (T_{CM}) cell phenotype, or at least 20% T_{CM} cells, or at least 30% T_{CM} cells, or at least 40% T_{CM} cells, or at least 50% T_{CM} cells, or at least 60% T_{CM} cells, or more than 65% T_{CM} cells.

The medium used in the method as described herein for stimulation and the medium used for expansion may contain the same concentrations of DMSO, in the range of 0.3-1.2% (v/v) DMSO, or may contain different concentrations of DMSO. Moreover, the concentration range of DMSO used in the media of the methods as described herein may be in the range of 0.2%-1.3% (v/v) DMSO, or more preferably in the range of 0.25%-1.25% (v/v) DMSO, or more preferably in the range of 0.3%-1.3% (v/v) DMSO, or more preferably in the range of 0.25%-1.3% (v/v) DMSO, or more preferably in the range of 0.3%-1.25% (v/v) DMSO, or more preferably in the range of 0.3%-1.1% (v/v) DMSO, or more preferably in the range of 0.3%-1.0% (v/v) DMSO, or more preferably in the range of 0.3%-0.9% (v/v) DMSO, or more preferably in the range of 0.3%-0.8% (v/v) DMSO, or more preferably in the range of 0.3%-0.6% (v/v) DMSO, or more preferably in the range of 0.3%-0.5% (v/v) DMSO, or more preferably in the range of 0.1%-1.3% (v/v) DMSO, or more preferably in the range of 0.1%-1.2% (v/v) DMSO, or more preferably in the range of 0.1%-1.0% (v/v) DMSO, or more preferably in the range of 0.1%-0.8% (v/v) DMSO, or more preferably in the range of 0.1%-0.6% (v/v) DMSO, or more preferably in the range of 0.1%-0.3% (v/v) DMSO, or more preferably in the range of 0.15%-1.3% (v/v) DMSO, or more preferably in the range of 0.15%-1.2% (v/v) DMSO, or more preferably in the range of 0.15%-1.0% (v/v) DMSO, or more preferably in the range of 0.15%-0.8% (v/v) DMSO, or more preferably in the range of 0.15%-0.6% (v/v) DMSO, or more preferably in the range of 0.15%-0.3% (v/v) DMSO, or more preferably in the range of 0.4%-1.3% (v/v) DMSO, or more preferably in the range of 0.4%-1.2% (v/v) DMSO, or more preferably in the range of 0.4%-1.0% (v/v) DMSO, or more preferably in the range of 0.4%-0.8% (v/v) DMSO, or more preferably in the range of 0.4%-0.6% (v/v) DMSO, or more preferably range of 0.5%-1.3% (v/v) DMSO, or more preferably in the range of 0.5%-1.2% (v/v) DMSO, or more preferably in the range of 0.5%-1.0% (v/v) DMSO, or more preferably in the range of 0.5%-0.8% (v/v) DMSO, or more preferably in the range of 0.5%-0.6% (v/v) DMSO, or more preferably range of 0.6%-1.3% (v/v) DMSO, or more preferably in the range of 0.6%-1.2% (v/v) DMSO, or more preferably in the range of 0.6%-1.0% (v/v) DMSO, or more preferably in the range of 0.6%-0.8% (v/v) DMSO, or more preferably in the range of 0.8%-1.3% (v/v) DMSO, or more preferably in the range of 0.8%-1.2% (v/v) DMSO, or more preferably in the range of 0.8%-1.0% (v/v) DMSO, or more preferably in the range of 1.0%-1.2% (v/v) DMSO, or more preferably in the range of 1.0%-1.3% (v/v) DMSO.

In a final aspect, pharmaceutical compositions comprising any of said human CAR-T cell compositions are disclosed herein. More particularly, one embodiment relates to a pharmaceutical composition comprising a human CAR-T cell mutated in MGAT5. In another embodiment said pharmaceutical composition comprises a human CAR-T cell mutated in MGAT5 that is devoid of tetra-antennary N-glycans at its cell surface.

Another embodiment relates to a pharmaceutical composition comprising the human CAR-T cell composition obtainable by the method described herein for production of a CAR-T cell composition with predominant memory phenotype, wherein said predominant memory phenotype is defined as said T cell composition comprising at least 10% of its cells being central memory T (T_{CM}) cells, or at least 20% T_{CM} cells, or at least 30% T_{CM} cells, or at least 40% T_{CM} cells, or at least 50% T_{CM} cells, or at least 60% T_{CM} cells, or more than 65% T_{CM} cells. Another embodiment relates to a pharmaceutical composition comprising the MGAT5 mutated human CAR-T cell composition obtainable by the method described herein for production of a human CAR-T cell composition with predominant memory phenotype, wherein said predominant memory phenotype is defined as said MGAT5 mutated human CAR-T cell composition comprising at least 10% of its cells being central memory T (T_{CM}) cells, or at least 20% T_{CM} cells, or at least 30% T_{CM} cells, or at least 40% T_{CM} cells, or at least 50% T_{CM} cells, or at least 60% T_{CM} cells, or more than 65% T_{CM} cells.

A further embodiment relates to any of the above pharmaceutical compositions further comprising additional components such as a diluent, excipient, preservatives or additive, as known in the art.

Another embodiment relates to any of the above pharmaceutical compositions for use as a medicament, preferably for use in cancer treatment, more preferably for use in treatment of a haematological malignancy or solid tumor. Another embodiment provides for any of said pharmaceutical compositions described herein for use in treatment of cancer, wherein said treatment prevents, inhibits, blocks or at least significantly reduces the incidence of relapse, or primary or secondary tumor growth and/or tumor burden.

It is to be understood that although particular embodiments, specific configurations, compositions, as well as materials and/or molecules, have been discussed herein for methods, compositions and products according to the disclosure, various changes or modifications in form and detail may be made without departing from the scope of this invention. The following examples are provided to better illustrate particular embodiments, and they should not be considered limiting the application. The application is limited only by the claims.

### ASPECTS OF THE DISCLOSURE

As described in the present application, the invention relates to a human CAR-T cell which has at least one genetic modification, which is a mutation in the N-acetylglucosaminyltransferase V (MGAT5) gene.

More specifically, the application relates to said MGAT5 mutated human CAR-T cell, which is devoid of tetra-antennary N-glycans at its cell surface.

Furthermore, the application relates to said MGAT5 mutated human CAR-T cell, for use in treatment of cancer, more specifically, wherein the cancer may be a haematological malignancy or solid tumor type of cancer.

A further embodiment relates to said MGAT5 mutated human CAR-T cell for use, wherein treatment prevents cancer relapse in a subject.

Further, said MGAT5 mutated human CAR-T cell may be used for treatment to prevent or inhibit secondary tumor growth and/or tumor burden in a subject.

A further embodiment relates to a method to produce a T cell composition with a predominant memory phenotype comprising the steps of:
(a) isolating T cells from a primary sample,
(b) incubating the T cell composition of step a) under stimulating conditions comprising 0.3-1.2% (v/v) Dimethylsulfoixde (DMSO), thereby generating a stimulated T cell composition, and wherein the T cell composition of step a) comprises at least a concentration of 1×10⁶ cells/mL,
(c) cultivating the stimulated T cell composition of step b) in a medium comprising 0.3-1.2% (v/v) DMSO for expansion of the T cell composition.

Said method may comprise a further step after step b) of: (b-bis) engineering the stimulated T cell composition by contacting the T cells with an agent comprising a polynucleotide encoding the recombinant receptor, preferably wherein the contacting is performed using a viral vector.

Alternatively said method may comprise a further step, after step b), and before, after or simultaneously with step b-bis) of (b-tris) introducing a mutation in T cells in the MGAT5 gene, optionally using CRISPR/Cas engineering.

Specifically, the application also describes the use of the method to produce a T cell composition for adoptive cell transfer to a subject.

Moreover, the application described the T cell composition obtainable by said method or the CAR-T cell composition obtainable by said method.

Any of said (CAR-)T cell compositions may be used for treatment of cancer, and/or specifically for treatment to prevent and/or inhibit relapse, primary or secondary tumor growth and/or tumor burden.

Finally, the application also describes a pharmaceutical composition comprising any of said human MGAT5 mutated CAR-T cells or (CAR-)T cell compositions described herein.

### EXAMPLES

### Introduction

As introduced De Bousser et al.¹, there is in vitro evidence that β-galactoside binding lectins (Galectins) can have a strong impact on the functionality of tumor-infiltrating T cells². The high-affinity poly-LacNAc N-linked galectin ligands are mainly synthesized onto the β1,6-GlcNAc branch introduced by N-acetylglucosaminyltransferase V (MGAT5) (FIG. 1). Knocking out MGAT5 should thus also strongly reduce the density of poly-LacNAc modifications on the cell surface. However, repeating LacNAc units can also be found on other types of glycosylation including O-glycans and glycolipids, and on the glycosaminoglycan keratan sulfate. This indicates that by knocking out MGAT5, we do not eliminate all potential galectin ligands.

By targeting this gene, the rest of N-glycan synthesis should remain intact. When targeting the locus encoding MGAT5 with Cas9 RNPs, high editing efficiencies can be obtained, as described further herein. In Examples 1-7, the impact of glyco-engineering through MGAT5 KO was evaluated on the CAR-T cell glycome and on its in vitro and in vivo activation, proliferation, differentiation and anti-tumor functionality. Moreover, in Examples 8-10, improved methods to engineer and produce (CAR-)T cell compositions are disclosed, revealing that specifically the presence of low concentrations of DMSO already at the stage of T cell stimulation provide for an increase in memory phenotype, improving the quality and transfection success when used in adoptive cell transfer and immunotherapy. The combination of applying MGAT5 mutated T cells for CAR-T therapy, and manufacturing those using the method applying DMSO during stimulation and expansion, may further increase the potency and efficacy of cancer treatment, especially in prolonging the memory towards blocking secondary/additional tumor initiation.

### Example 1. Engineering of MGAT5 KO CD70 nanoCAR-T Cells

To evaluate the impact of altered cell surface glycosylation on cytotoxic T cell functionality, specifically in a cancer immunotherapy setting, we chose CD70 as the CAR-Target. Nanobodies targeting CD70 have been generated previously. Subsequently, these nanobodies have been thoroughly evaluated as antigen-binding module in a CAR format (CD70 nanoCAR; data not shown).

A workflow for a combined CRISPR-Cas9 mediated glyco-gene editing and retroviral CAR delivery to purified activated human CD3⁺ T cells was setup. The presence of both CD4′ and CD8⁺ T cell subsets in the final CAR-T cell product is indispensable for efficient anti-tumor immunity. Optimal editing and transduction efficiencies were obtained when CD3⁺ T cells were stimulated with Immunocult for three days, after which activated T cells were first subjected to Cas9 ribonucleoprotein (RNP) nucleofection (comprising the guide RNA of SEQ ID NO:1 to target the MGAT5 locus), followed by a 1-hit retroviral transduction on the same day. Engineering efficiencies were assessed on day 10. The experimental timeline is depicted in Error!Reference source not found.A.

For in vitro analyses described herein, cells were cultured in IL-2 in order to study T cell effector functions. In contrast, for in vivo studies described herein, cells were cultured in IL-7 and IL-15 following stimulation in the presence of IL-12 to obtain cells with a desired phenotype for adoptive cell transfer (ACT) studies (mostly naïve T cells).

CRISPR editing efficiencies were determined by Sanger sequencing of the region of interest followed by ICE analysis, and the mean editing efficiency as percentage insertions and deletions (% indel) for the MGAT5 locus over multiple experiments was consistently high (exceeding 80% indel) as is depicted in Error!Reference source not found.B. Flow cytometry was used to measure both CD70 nanoCAR expression and GFP expression as read outs of the retroviral transduction efficiency. High CD70 nanoCAR-Transduction efficiencies were consistently obtained over multiple experiments, irrespective of the simultaneous glyco-gene engineering as shown in Error!Reference source not found.C.

### Example 2. Evaluation of the Structural Impact of MGAT KO on the CD70 nanoCAR-T Cell Glycocalyx

In order to be able to assess the extent of the intended glycosylation changes upon glyco-gene engineering, we developed a lectin-based flow cytometry assay. For the detection of poly-LacNAc structures, the lectin from Datura stramonium (DSL) was included Error!Reference source not found.A. This lectin is reported to bind well to N-acetyllactosamine and oligomers containing repeating N-acetyllactosamine sequences next to its preferred N-acetylglucosamine oligomer ligand.

When comparing the DSL lectin stain intensity for mock engineered CD70 nanoCAR-T cells with that of MGAT5 KO CD70 nanoCAR-T cells, we observe a clear reduction in signal, indicating that we successfully eliminated N-glycan β-1,6-branching and subsequent elongation of this branch with poly-LacNAc modifications.

As a complementary method to profile the CAR-T cell surface glycosylation, the DSA-FACE method was adapted to enable the analysis of cell surface N-glycosylation. We aimed to directly release the N-glycans from the cell surface by applying the PNGaseF digest on living cells in suspension. An optimized protocol was established in which we incubate 1×10⁶ cells per sample in the presence of 0.125 IU PNGaseF in PBS for 2 hours at 37° C. Subsequently, the cells are removed by centrifugation and the crude digest is labeled with APTS for 1 hour at 70° C. After two rounds of clean-up over Sephadex resin to remove excess label and salts, labeled N-glycans are resuspended in water and analysed by DSA-FACE. The flow is schematically depicted in Error!Reference source not found.B. When CAR-T cells are engineered for MGAT5 knock out (Error!Reference source not found.B), the N-glycan profile is clearly different from that of mock-engineered CAR-T cells. The peaks in P6 disappear while the peaks in P4 show a higher intensity relative to P2 and P3. This shift in electrophoretic mobility is consistent with the removal of one LacNAc unit (two monosaccharide units) or a shift from a tetra-antennary to a tri-antennary N-glycan. These DSA-FACE results are also in agreement with the lectin-staining experiments, where we observed a reduction in DSL staining intensity upon MGAT5 engineering (Error!Reference source not found.A). When comparing to the annotated N-glycan profile of human plasma (data not shown), this observation indeed confirms that P6 corresponds to a tetra-antennary N-glycan, while peaks in P4 and P5 correspond to tri-antennary N-glycan structures.

### Example 3. Characterization of the CD70 Expressing Tumor Cell Lines

In order to study the anti-tumor functionality of the glyco-engineered CD70 nanoCAR-T cells, two tumor cell lines were used. THP-1 cells are a M4 subtype acute myeloid leukemia (AML) cell line and SKOV-3 cells are a serous adenocarcinoma cell line. We confirmed the cell surface CD70 expression on these cells by flow cytometry (Error!Reference source not found.A). Jurkat cells (immortalized line of human T cells) were included as a negative control. Further, we performed the anti-CD70 cell surface staining on non-transduced (NTC) and CD70 nanoCAR-Transduced CD3⁺ T cells and did not identify auto-antigen expression.

Galectins exert a broad range of effects during different aspects of T cell-mediated immunity by the formation of lattices on the T cell surface. In anti-tumor immunity, it has been shown that Galectin-1 and Galectin-3 in the TME lead to tolerogenic signaling and immune suppression. N-acetyllactosamine is the ligand recognized by Galectins and the affinity of the interaction is proportional to the N-acetyllactosamine content of the glycan structure. We hypothesized that by eliminating MGAT5 expression in order to reduce the poly-LacNAc content on cytotoxic T cells, the inhibitory effect of Galectins on T cell immunity can be reduced. To this end, we aimed to evaluate whether the tumor cell lines used in our study indeed express Galectin-1 and Galectin-3.

Secretion and subsequent cell surface binding of Galectin-1 and -3 was detected by performing a flow cytometry experiment with anti-Galectin-1 and -3 antibodies. The results are shown in Error!Reference source not found.B. As positive control, cells were incubated with recombinant Galectin-1 or -3 before performing the cell surface staining. As a negative control, cell surface Galectin binding was abolished by the addition of the competitive inhibitor lactose. Jurkat cells were included as negative control. Galectin-1 expression is detected for both the THP-1 and SKOV-3 cell lines. Further, galectin-3 expression is clearly observed for the SKOV-3 cell line and less for the THP-1 cell line. No secretion and cell surface binding of galectins is seen on primary CD3⁺ T cells and Jurkat cells. Binding of recombinant Galectin-3 to T cells leads to an increase in signal, while recombinant Galectin-1 does not seem to bind to the primary T cells.

Additionally, we confirmed the expression of Galectin-1 and -3 in tumor sections from tumor-bearing NSG mice (Error!Reference source not found.C). the latter were obtained by ectopically inoculating human SKOV-3 cells. The SKOV-3 tumor model is used and explained in more detail in the experiments described below.

### Example 4. The Impact of Glyco-Engineering on In Vitro CD70 nanoCAR Functionality

The impact of altered glycosylation due to MGAT5 KO on the in vitro functionality of CD70 nanoCAR-T cells is analyzed herein. CD70 nanoCAR-T cells with an MGAT5 KO were engineered starting from CD3⁺ T cells. Cells were cultured and expanded in the presence of IL-2 until day 13, the day that the in vitro functional tests are initiated. High viability is maintained for each condition as is depicted in FIG. 2. A bias towards CD8⁺ T cells is noted in non-transduced cells (NTC), since IL-2 was added to the culture medium to promote CD8⁺ T cell growth. Interestingly, the addition of IL-2 does not increase the fraction of CD8⁺ T cells in CD70 nanoCAR expressing T cells as significantly as in the control cells. Even with the 4-1BB signal, which is believed to support a moderate rise in the CD8⁺ T cell fraction, the CAR-T cell groups show a decrease in the CD8′ population. Furthermore, this decrease is even more pronounced when CD70 nanoCAR-T cells were CRISPR-Cas9 engineered, which suggests that the viral transduction and nucleofection procedures might affect the growth of CD8⁺ T cells more than that of CD4⁺ T cells (FIG. 2).

The Impact of Glyco-Engineering on In Vitro CD70 nanoCAR Cytokine Production.

The antitumor effects of CAR-T cells depend on their capacity to secrete cytokines upon exposure to antigens. Therefore, we evaluated the cytokine production of the glyco-engineered CD70 nanoCAR-T cells after challenging them with the THP-1 and SKOV-3 target cell lines. Target cells were co-incubated for 16 hours with MGAT5 KO CD70 nanoCAR-T cells. Unstimulated cells were included as negative control (−) and Immunocult stimulation was included as positive control (+). Subsequently, T cells were labelled for CD4 and CD8 and intracellular TNF-α, IFN-γ and IL-2. The results are shown in Error!Reference source not found. The glyco-engineered CD70 nanoCAR-T cells are able to produce cytokines upon antigen stimulation and the proportion of positive cells is similar to, or even higher than what is observed for CD70 nanoCAR and mock nucleofected CD70 nanoCAR-T cells. This cytokine expression is dependent on CD70 nanoCAR expression, given that non-transduced T cells (NTC) fail to express cytokines in the presence of CD70 positive cells (but do show expression of cytokines after polyclonal Immunocult stimulation).

The Impact of Glyco-Engineering on In Vitro CD70 nanoCAR Cytotoxic Potential.

In order to evaluate the cytotoxic potential of glyco-engineered CD70 nanoCAR-T cells, T cells were co-cultured with THP-1 target cells at different ratios for a period of 14 days. The number of THP-1 cells left in culture was determined by flow cytometry every three to four days. At day 7, a second challenge was performed by adding target THP-1 cells to the co-cultures. Representative results for two independent experiments are depicted in Error!Reference source not found. Error!Reference source not found.A shows the results corresponding to an Effector/Target (E/T) ratio of 0.15, that is 20 000 THP-1 target cells co-cultured with 3000 CD70 nanoCAR effector cells. At this ratio, all target cells get killed by day 3, in the wild-type, mock engineered and MGAT5 KO CD70 nanoCAR-T cell conditions. Even at a very low E/T ratio of 0.015 (20 000 target cells co-cultured with only 300 CD70 nanoCAR-T cells), all target cells are eliminated by day 14, irrespective of the engineering condition (Error!Reference source not found.B). In general, glyco-engineered CD70 nanoCAR-T cells behave similarly to mock nucleofected CD70 nanoCAR-T cells.

As an extension to these results, CD70 nanoCAR T cells were engineered and cultured in the presence of IL-7 and IL-15 using the protocol used for in vivo experiments. Data were collected in the same manner as described above and represented in FIG. 19. A similar efficacy was observed for THP-1 killing over time for both E/T ratios when comparing mock nucleofected CD70 nanoCAR-T cells and glyco-engineered CD70 nanoCAR T cells. Most importantly, a higher proliferation of CD70 nanoCAR T cells was observed over time for the MGAT5KO cells.

### Example 5. The Impact of Glyco-Engineering on In Vivo CD70 nanoCAR Functionality

After validating the in vitro activity of the MGAT5 KO CD70 nanoCAR-T cells, we aimed to evaluate whether MGAT5 KO CD70 nanoCAR-T cells are also capable of clearing a tumor upon adoptive transfer in vivo. A schematic representation of the experimental timeline is depicted in FIG. 8 and FIG. 20A.

The NOD.SCID IL2rγ^{null} (NSG) mouse strain has been widely used in the pre-clinical evaluation of CAR-T cell efficacy. Immune-deficient NSG mice lack functional mouse T cells, B cells, NK cells and are deficient in cytokine signaling through the common γC receptor⁴. Human tumor xenograft models were established in NSG mice by subcutaneous injection of luciferase expressing SKOV-3 cells in the flank. Ten days after tumor cell inoculation, the presence of a subcutaneous tumor was evaluated by measurement with a slide caliper and through bioluminescent imaging (BLI) performed using an in vivo imaging system (IVIS).

Glyco-engineered MGAT5 KO CD70 nanoCAR-T cells were generated as described above and cultured in the presence of IL-7 and IL-15 until day 13. After establishment of a solid, palpable tumor, mice were treated with either mock Cas9-engineered or MGAT5 KO CD70 nanoCAR, respectively. As control groups, mice were treated with PBS to evaluate tumor development, or with non-transduced T cells (NTC) to evaluate graft versus host disease (GvHD) and non-specific anti-tumor effects. Throughout the experiments, tumor burden was measured every two days with a caliper and every 4 days through IVIS. A schematic representation of the experimental timeline is depicted in Error!Reference source not found.8 (Experiment A) and 20A (Experiment B).

At day 34, after the first phase of the experiment, the presence and phenotype of CAR T cells was evaluated in the blood and/or spleen. Furthermore, mice were followed-up in time and challenged between day 85/87 or day 90 with a second tumor to evaluate long-term anti-tumor efficacy. Again, tumor burden was evaluated over time and the mice were sacrificed between day 116/118 or 123 for end-point analyses.

Results of the initial in vivo analyses (Experiment A) are summarized in Error!Reference source not found.-Error!Reference source not found. Tumors were completely eliminated in mice treated with 2.5×10⁶ mock Cas9 and MGAT5 KO CD70 nanoCAR-T cells between day 20 and day 31 (Error!Reference source not found.A-C). End-point analysis on day 34 was performed on peripheral blood and spleen by flow-cytometry (Error!Reference source not found.D-I). Human CD3⁺ T cells were detected in blood and spleen of mice treated with mock Cas9 or MGAT5 KO CD70 nanoCAR-T cells and around 75% of these cells were found to be CD70 nanoCAR-T cells (data not shown), based on GFP expression. The number of MGAT5 KO CD70 nanoCAR-T cells in the spleen is markedly increased as compared to mock Cas9 CD70 nanoCAR-T cells (Error!Reference source not found.G). The immunophenotype of mock Cas9 and MGAT5 KO CD70 nanoCAR-T cells was father analyzed (Error!Reference source not found.E, F, H and I). Both in blood and the spleen, the majority of the CD70 nanoCAR-T cells (over 80%) were CD4⁺. Most of the CD70 nanoCAR-T cells were found to have an effector phenotype. Five mice that were treated with CD70 nanoCAR-T cells or MGAT5 KO CD70 nanoCAR-T cells were followed for a prolonged period of time to ensure that the original tumor did not regrow on the site of initial inoculation (right flank). By day 80, all mice remained tumor-free as demonstrated in Error!Reference source not found.A. To evaluate the engraftment of CAR-T cells, blood was withdrawn from the tail vein on day 80 and human T cells and CAR-T cells were detected using flow cytometry. The results are depicted in Error!Reference source not found.B-D. Human CD3⁺ T cells were still present in the blood at day 80 and around 45% of these cells were CD70 nanoCAR-T cells (evaluated based on GFP expression) (Error!Reference source not found.B). The majority of the CD70 nanoCAR-T cells were CD4⁺ T cells (Error!Reference source not found.C) in an effector memory differentiation state (Error!Reference source not found.D). At day 85, a second challenge with tumor cells was performed by inoculation of SKOV-3 cells at the left flank of the mice to verify whether these CD70 nanoCAR-T cells were still effective in eradication of the tumor. Naïve mice of the same age did receive the same amount of SKOV-3 cells, and served as a control group. From day 89 onwards, body weight and tumor burden were followed as described before. The results are depicted in Error!Reference source not found. Body weight of the mice throughout the rechallenge experiment did not significantly alter, but our observations indicate that the tumor burden is markedly lower in mice previously treated with MGAT5 KO CD70 nanoCAR-T cells as compared to the previously untreated and mock-engineered CD70 nanoCAR-treated group (Error!Reference source not found.A and B). End-point analysis on day 116 was performed on peripheral blood and spleen by flow-cytometry (Error!Reference source not found.C-H). Human CD3⁺ T cells were detected in blood and spleen of mice treated with mock Cas9 or MGAT5 KO CD70 nanoCAR-T cells and around 75% of these cells were found to be CD70 nanoCAR-T cells (data not shown), based on GFP expression. The immunophenotype of mock Cas9 and MGAT5 KO CD70 nanoCAR-T cells was further analyzed (Error!Reference source not found.D, E, G and H). Both in blood and the spleen, the majority of the CD70 nanoCAR-T cells (over 80%) were CD4⁺. Most of the CD70 nanoCAR-T cells were found to have an effector phenotype in the spleen, or an effector or effector memory phenotype in the blood.

Secondly, a further independent experiments (Experiment B) was performed with T cells from different donors, similarly to the experiment above (FIG. 20A). For the analysis, the treatment groups were divided in three sets of interest: The ‘No CAR’ dataset contains the data from all the mice that did not receive any CD70 nanoCAR T cells, and thus includes untreated mice and mice treated with PBS or NTC. The ‘CAR’ dataset contains the data from all the mice that received a CD70 nanoCAR T cell treatment, with or without mock Cas9 engineering. The ‘CD70 nanoCAR-MGAT5 KO’ dataset contains data from the mice that received MGAT5 knockout CD70 nanoCAR T cells.

The outcome of the treatment was defined by 4 subtypes for the primary tumor challenge. (1) Full control meaning the tumor becomes undetectable and no relapse follows. (2) Full control but occurrence of a relapse later on. (3) Partial control meaning a halt in tumor growth but the tumor remains detectable and all mice also experience a relapse after long-term follow-up. (4) No control of tumor growth throughout the duration of the experiment.

As is clear from Error!Reference source not found.20B and the table in Error!Reference source not found.20C, the primary tumor is not controlled by the mice that did not receive CAR-T cells, meaning that they were all sacrificed at the humane end-point. When we compare CD70 nanoCAR treated groups with MGAT5 KO CD70 nanoCAR treated groups, we see that more mice control tumor growth when they were treated with MGAT5 KO CD70 nanoCAR T cells, and that none of these mice show no or only partial control of tumor growth, while this is the case for a considerable group in the CAR treated groups.

As opposed to experiment A, in which we did not observe any relapse of the primary tumor over time, the tumor did regrow in some of the treated mice in experiment B. A survival analysis was performed to evaluate whether a difference could be observed in either the number of relapses and the time of their onset between Mock Cas9 engineered and MGAT5 KO CD70 nanoCAR T cell treated mice. When we looked into the Kaplan-Meier curves, we indeed observed a difference. The CD70 nanoCAR group seemed to have more relapses and earlier onset in time, as well with a median tumor free survival time of 55 as compared to the MGAT5 KO CD70 nanoCAR treated group in which the median tumor free survival time is 72 (FIG. 22). However, this result is not statistically significant, which is probably due to the low number of mice followed-up at this point.

We ran a multinomial logistic regression model (with proportional odds assumption) on the data of the two experiments combined, to test whether the response to treatment was ameliorated in mice that received MGAT5 KO CD70 nanoCAR T cells in comparison to those that received non-glyco-engineered CD70 nanoCAR T cells. We found that, in the MGAT5 KO CD70 nanoCAR T cell treated group, the estimated odds of having an outcome that is better than a given level is an estimated 5.5 times higher than in the CD70 nanoCAR treated group (95% CI: 1.06 to 28.52). This further supports the observation that the mice were doing better with MGAT5 KO CD70 nanoCAR T cell treatment, but the p-value is only just significant (0.042).

For the secondary tumor, we defined three types of tumor control as no relapse of tumor growth was observed in any of the mice that cleared the secondary tumor. (1) Full control meaning the tumor never develops or becomes undetectable after an initial growth phase. (2) Partial control meaning the tumor stops growing but remains detectable. (3) No control of tumor growth throughout the duration of the experiment.

As is clear from Error!Reference source not found.20B and the table in Error!Reference source not found.20C, MGAT5 knockout CD70 nanoCAR T cell treatment also leads to better tumor control after a secondary challenge. As the majority of the mice experience no or only partial control of the secondary tumor in the CAR treated groups (52.9% in total), this image is shifted in the MGAT5 KO CAR treated groups (35% in total). In the latter, the majority of the mice completely clear the tumor before the end of the experiment (64% of the mice in total). In the mice that did not clear the secondary tumor completely, the majority of mice treated with the MGAT5 knockout CD70 nanoCAR T cells experienced partial control (21%) while the majority of mice treated with wild type CD70 nanoCAR T cells showed no control at all (47%). We again used the model to test whether there is a difference between the CD70 nanoCAR T cell treated groups and MGAT5 KO CD70nanoCAR T cell treated groups. In this case, there was not enough evidence in the model to say that the MGAT5 KO CD70 nanoCAR T cell treatment leads to a better outcome (odds ratio of 3.22 but with a wide confidence interval (95% CI: 0.73 to 14.18) with a p-value of 0.122. This is probably due to too little measurement points in the dataset.

MGAT5 KO CD70 nanoCAR T Cells are Present in Higher Numbers than CD70 nanoCAR T in Peripheral Blood and Spleen Following Tumor Control.

End-point analysis on day 34 was performed on peripheral blood (both experiments) and spleen (experiment A) by flow-cytometry (FIG. 21-A,B,F). Human CD3⁺ T cells were detected in blood and spleen of mice treated with mock Cas9 or MGAT5 KO CD70 nanoCAR T cells and around 75% of these cells were found to be CD70 nanoCAR T cells (data not shown), based on GFP expression. The number of MGAT5 KO CD70 nanoCAR T cells in the spleen (FIG. 21-B) and blood (FIG. 21-F) is markedly increased as compared to mock Cas9 CD70 nanoCAR T cells. We did not analyze splenocytes on day 34 in experiment B, since we kept all mice for rechallenge, enabling statistics on larger groups. CAR T cells were still present in the blood at day 80 (FIG. 21-C,G). We see a trend of higher numbers of MGAT5 KO CD70 nanoCAR T cells compared to CD70 nanoCAR T cells, mostly pronounced in experiment B, however, the difference is not statistically significant. End-point analysis between day 118 and day 123 was performed on peripheral blood and spleen (FIG. 21-E,I). Similarly, in both experiments we measure higher numbers of MGAT5 KO CD70 nanoCAR T cells compared to CD70 nanoCAR T cells, however, the difference is not statistically significant.

### Example 6. Treatment of Tumor-Bearing Mice with MGAT5 KO CAR T Cells Leads to a Better Control of Tumor Growth Rate

To evaluate differences in tumor growth between the treated mice of Example 5 (Exp A & B; FIGS. 20-21), a piecewise linear mixed model (with interactions) was fitted (see FIG. 23-27) that allows to model the mean traces of each group. For these analyses, we made a distinction between Experiment A and Experiment B. The main reason for this is that the model would become unnecessarily complex because the timescales (design) of both experiments differ slightly as do the times at which the mice start to respond to the CAR T cell therapy. The latter is possibly due to inherent differences between the T-cell batches.

In experiment A, we did not observe a difference in the growth rate of the primary tumor in mice treated with CD70 nanoCAR T cells and those treated with MGAT5 KO CD70 nanoCAR T cells (FIG. 23Error!Reference source not found.). However, when we look at the response to treatment in the secondary tumor phase (FIG. 24Error!Reference source not found.), differences were observed. While the tumor in the untreated mice grows at 2% per day (which is consistent with the primary tumor growth), the average growth rate in the CD70 nanoCAR T cell treated group is 3% per day. However, the tumor size in the MGAT5 KO CD70 nanoCAR T cell treated group decreases with 10% each day, indicating that MGAT5 KO CD70 nanoCAR T cells control tumor growth rate more efficiently in the secondary phase. However, due to the diversity of individual mice in these groups, the confidence intervals on these measurements are wide, and the observation is therefore not statistically significant with a 95% confidence interval of 0.67 to 1.13 (Adj. p-value of 0.505).

In experiment B, we already observe a difference in the average tumor volume when we compare CD70 nanoCAR treated mice with those that received MGAT5 KO CD70 nanoCAR T cells (FIG. 25). Between day 22 and day 33, the MGAT5 KO CD70 nanoCAR T cells clear the tumor faster. The tumor growth rate in this group is only 71% of that in the CD70 nanoCAR T cell treated group which is statistically significant (CI of 0.59-0.86, adj. p-value of <0.001). Moreover, the average tumor volume is also significantly smaller in the MGAT5 KO CD70 nanoCAR T cell treated group between day 33 and day 84. However, the confidence interval is quite wide, probably due to the large spread in the CD70 nanoCAR T cell treated group, were some mice clear the tumor completely, some partially and some not at all. Unlike what was observed in experiment A, some of the primary tumors did regrow in the course of the experiment B. From the analysis shown in FIG. 26, it is clear that both the average tumor volume and the growth rate are significantly lower in the MGAT5 KO CD70 nanoCAR T cell treated group, as compared to the CD70 nanoCAR T cell treated mice.

The response of CD70 nanoCAR T cell therapy on a secondary tumor challenge in experiment B is summarized in FIG. 27. From day 101 onwards, we see that the tumor size in the MGAT5 KO CD70 nanoCAR T cell treated group decreases with 10% each day, while the tumor size in the untreated and CD70 nanoCAR T cell treated groups increases with 9% and 6% daily respectively, again indicating that MGAT5 KO CD70 nanoCAR T cells lead to a better tumor control after a secondary challenge. However, this result is not statistically significant with a 95% confidence interval of 0.66 to 1.23. This is most probably due to the large variability and relatively few available mice within the treatment groups.

### Example 7. The Impact of Reduced N-Acetyllactosamine Modifications on CAR-T Cell Behavior

Our results indicate that MGAT5 KO CD8⁺ and CD70 nanoCAR-T largely behave in the same way as control cells in vitro. MGAT5 elimination had no clear impact on T cell activation or exhaustion markers, proliferation and viability. Furthermore, in vitro and in vivo anti-tumor cell responses were maintained. Notably, increased numbers of MGAT5 KO CD70 nanoCAR-T cells were observed, both in vitro and in vivo, as demonstrated in examples 5 and 6.

It was previously shown that the inhibition of N-acetyllactosamine glycans via competitive inhibition with carbohydrate analogs increased the number of infiltrating tumor-specific T cells⁵. In Ye et al.⁶, MGAT5 was identified in a CRISPR screen in murine CD8⁺ T cells in a syngeneic model of glioblastoma in immunocompetent mice. MGAT5 perturbation enhanced the efficacy of adoptive T cell transfer against glioblastoma in mice with both immunocompetent and antigen-specific transgenic TCR models in terms of increased tumor infiltration and overall survival of tumor bearing mice. An increased number of MGAT5 KO CD70 nanoCAR-T cells compared to control CD70 nanoCAR-T cells has been shown herein, both in vitro and in vivo. MGAT5 KO CAR-T cells may thus be less susceptible to Galectin-3-mediated apoptosis, since Galectin-3 overexpression was demonstrated in the tumor cell lines used in our models and are analyzing whether Galectin-3 binding to MGAT5 KO (CAR) T cells is indeed reduced. To this end, T cells are incubated with recombinant galectin and subsequently stained with anti-galectin antibodies. Furthermore, MGAT5 KO and WT (CAR) T cells are incubated with recombinant Galectin-3 to assess whether the elimination of Poly-LacNAc structures has the ability to block galectin-mediated T cell apoptosis ⁷.

In order to study glyco-engineered CAR-T cell functionality in the presence of a reconstituted human immune system, the aforementioned THP-1 tumor model is setup in HIS (humanized immune system) mice. Mice are humanized by injections of human CD34⁺ umbilical cord stem cells into the liver of neonatal mice, leading to the engraftment of these cells and development into human lymphocytes⁹.

Further validation is performed in an appropriate set of patient-derived xenograft (PDX) models. PDX models are developed by transplantation of freshly isolated patient-derived tumor fragments in immunodeficient mice. These models better recapitulate the primary tumor immunosuppressive environment^{10,11}. PDX models are increasingly recognized as clinically relevant preclinical models for oncology research because they offer greater predictive value than traditional cell line models. Furthermore, they preserve the heterogeneous pathological and genetic characteristics of original patient tumors and provide a more translatable response.

### Example 8. Method for Producing (CAR) T Cell Compositions Skewed Towards a Memory Phenotype

(CAR) T cell production parameters and conditions impact the quality and potential successful engrafting of the final composition when used for adoptive immune therapy. Generally, it has been established that T cells with a memory phenotype exert superior anti-cancer immune responses.

DMSO was shown to have an in vitro anti-inflammatory effect by reducing lymphocyte activation, revealed by reduced proliferation and decreased cytokine production (TNF-α, IFN-γ and IL-2)^{24,26}. The immunosuppressive activity of DMSO was also demonstrated using in vitro and in vivo models by Lin et al.²⁷. They showed that addition of DMSO decreases the percentage of IFN-γ-producing T lymphocytes and increases the percentage of Treg cells by enhancing the activation of the STAT5 signaling pathway in the context of autoimmune diabetes. Also in this regard, DMSO was shown to stimulate the production of transforming growth factor-β (TGF-β), an immunosuppressive cytokine, as well as the recruitment of its receptor from intracellular vesicles to the plasma membrane²⁸. Even at very low concentrations (0.1-1%), DMSO seems to suppress CD4⁺ T cell activation and cell growth as well as cytokine secretion²⁹. Further, it has been shown that treatment of pluripotent stem cells (PSCs) with a low concentration of DMSO significantly increases the propensity of a variety of PSCs to differentiate to different cell types following directed differentiation. The DMSO pre-treatment improves differentiation by regulating the cell cycle and priming stem cells to be more responsive to differentiation signals³⁰⁻³².

Following initial observations that suggested a possible effect of DMSO that was used as a vehicle present in in vitro cell cultures of CD8⁺ T cells, we were triggered to better understand the effect of low doses of DMSO on human CD8⁺ T cell proliferation and differentiation characteristics. These DMSO concentrations are generally used in assays that measure T cell responses and function, and where antigens and other proteins are added.

Next, we describe a method for in vitro culturing of T cells in the presence of different cytokine combinations and with addition of a concentration range of low amounts of DMSO, to analyze the T cell populations in in terms of survival, proliferation, activation, exhaustion and differentiation.

### Low Concentrations of DMSO do not Impact CD8. T Cell Survival, Proliferation and Activation.

Human CD8⁺ T cells were purified and incubated with human T cell-activator CD3/CD28 dynabeads for T cell expansion and activation, and this in the presence of different cytokine combinations and increasing concentrations of DMSO. In order to evaluate the effect of DMSO on the proliferation of T cells in culture, cells were labeled with Tag-it Violet™ proliferation and cell tracking dye. Tag-it Violet™ passively diffuses into the cell where esterases cleave acetoxymethylesters on the molecule. Tag-it Violet™ then covalently attaches to intracellular proteins enabling its long-term retention. Flow cytometry analysis was performed on multiple days following start up until day 11 in culture in order to evaluate T cell activation and differentiation markers. In a similar set-up, the effect of increasing concentrations of DMSO on in vitro mouse CD8⁺ T cell activation and differentiation was evaluated, providing similar results.

From FIG. 12 it is clear that the presence of DMSO in human T cell cultures (up to a concentration of 1.2%) does not have a negative effect on survival of the cells, cultured in the presence of either IL-2 (panel A) or IL-7/15 (panel B). Lower concentrations of DMSO (0.15-0.6%) even seem to protect the cells, since we see small increases in survival rate, especially in the IL-2 condition. Along the same line, we do not see major effects of DMSO on proliferation of CD8⁺ T cells. When cells are cultured in the presence of IL-7 and IL-15 for a prolonged period of time (FIG. 12B), an inhibitory effect of DMSO on T cell proliferation is observed when 0.6% DMSO or more is added to the cell culture. However, when cells are cultured in the presence of IL-2, a concentration of 0.6% is still tolerated and only the 1.2% DMSO conditions shows marked decrease in T cell proliferation over time (FIG. 12A).

The activation markers CD69 and CD25 are highly expressed on cells activated with Dynabeads (CD25⁺ and CD69⁺ cells on day 4, FIG. 12). The frequency of cells that are CD25 positive decreases in time and with increasing DMSO concentration, while the proportion of CD69 positive cells increases with increasing concentrations of DMSO. Further, the expression level of CD25 on T cells is lower in DMSO treated cells as compared to untreated cells while the expression of CD69 is higher as is clear from the representative histograms in FIG. 12 showing the fluorescence intensity.

It has been described that a small fraction (1-20%) of circulating memory T cells express CD69, the latter mediating homing and retention of memory T cells in the bone marrow³³. We hypothesize that the increased proportion of CD69 expressing cells seen with increased concentration of DMSO, might be indicative of an increase in memory phenotype as further described below.

### Expression of the Negative Immune Regulator TIM-3 is Downregulated on CD8⁺ T Cells in the Presence of Increasing DMSO Concentrations.

A number of pathways and molecules have been described in mediating T-cell dysfunction and exhaustion, of which programmed cell death-1 (PD-1), cytotoxic T lymphocyte antigen-4 (CTLA-4) and mucin domain-containing protein 3 (TIM-3) are of the most studied ones. We have analyzed the expression of these exhaustion markers on CD8⁺ T cells in culture in the presence of increasing concentrations of DMSO (FIG. 13). PD-1 and CTLA-4 are markedly upregulated after stimulation (day 4) and expression levels decrease over time. Although most CD8⁺ T cells are negative for PD-1 and CTLA-4 by day 11 in culture, the proportion of cells expressing PD-1 after stimulation seems to decrease faster by day 7 when cells are cultured in the presence of IL-2 and increasing concentrations of DMSO (FIG. 13A). The expression of TIM-3 remains high during the entire culturing period of human CD8⁺ T cells in either cytokine condition. This is in line with previous observations showing that TIM-3 is induced by the common γ-chain cytokines IL-2, IL-7, IL-15 and IL-21 in an antigen-independent manner and is upregulated on primary T cells in response to T-cell receptor and CD28 stimulation³⁴. We do see a decrease in the proportion of CD8⁺ T cells expressing TIM-3 in the presence of increasing concentrations of DMSO although the decrease is small (+/−4% in the presence of IL-2 and +/−10% in the presence of IL-7 and IL-15) (Error!Reference source not found.13B). Moreover, as is clear from the representative histograms showing fluorescence intensities, the TIM-3 expression level is markedly reduced in the presence of DMSO.

When evaluating the expression of activation markers, we observed a decrease in CD25-expressing cells while CD69 expression was elevated with increasing DMSO concentrations. Further, a decrease in TIM-3 expression was observed upon culturing in the presence of DMSO, indicating a less exhausted T cell phenotype.

### Example 9. DMSO Skews T Cell Differentiation Towards a Memory Phenotype

Human T cell differentiation subsets were evaluated during culturing in the presence of different concentrations DMSO and identified as naive CD8⁺ T cells (CD45RA⁺CD62L⁺ or CD45RA⁺CCR7⁺), effector CD8⁺ T cells (CD45RA⁺CD62L⁻ or CD45RA⁺CCR7⁻), effector memory CD8⁺ T cells (CD45RA⁻CD62L⁻ or CD45RA⁻CCR7⁻) and central memory CD8⁺ T cells (CD45RA⁻CD62L⁺ or CD45RA⁻CCR7⁺). Increasing concentrations of DMSO induced a decrease in naive and effector CD8⁺ T cells in favor of an increase in memory T cells encompassing central memory as well as effector memory T cells. This effect is most clear in the presence of IL-2, in which untreated cells mainly have a naive phenotype by day 11, while DMSO treated cells shift towards a memory phenotype (FIGS. 14A and B). The skew to a memory phenotype does also occur in the presence of IL-7 and IL-15 (FIGS. 14C and D). In this case, cells preferentially differentiate towards a T_{CM} phenotype while a more even distribution between T_{EM} and T_{CM} memory T cells is observed when cells are cultured in the presence of IL-2.

Altogether, our data suggest an effect for DMSO in skewing the differentiation status of human CD8⁺ T cells towards a memory-like phenotype irrespective of the cytokine conditions adopted to culture the T cells and these observations were confirmed for mouse CD8⁺ T cell cultures.

### Example 10. The Addition of DMSO to Human CD3⁺ T Cell Cultures Skews CD8⁺ T Cell Differentiation Towards a Memory Phenotype

We already described the importance of the presence of both CD4⁺ and CD8⁺ T cell subsets in therapeutic T cell products. Further, it was shown that memory T cells exert superior anti-tumor functions. A protocol for in vitro isolation of CD3⁺ T cell population used herein includes stimulation of purified human CD3⁺ T cells in the presence of IL-12 and culturing in the presence of either IL-2 or IL-7/-15. This cultivation method provides for CD3⁺ T cells populations skewed towards a naive phenotype. Our observations of the impact of DMSO on the differentiation state of CD8⁺ T cells led to the hypothesis that we might be able to tweak the naive differentiation state of cultured CD3⁺ T cells towards a more memory-like phenotype in the presence of DMSO. To this end, we validated the effect of DMSO on the activation and differentiation of CD3⁺ T cells in the presence of IL-12 and subsequent culturing with different cytokine combinations.

CD3⁺ T cells engineered ex vivo for the study of immune therapeutic applications are generally cultured in vitro for a duration of two weeks prior to functional analyses or therapeutic use in the context of CAR T cells. We therefore evaluated the impact of increasing DMSO concentrations on the immunophenotype at day 14, and the impact of the highest DMSO concentration (1.2%) on the immunophenotype of CD3⁺ T cell (sub)populations at day 13.

Increasing DMSO concentration did not impact CD3⁺ T cell survival and proliferation, irrespective of the cytokine combination (FIG. 15). In the presence of IL-2, we observe a higher proportion of CD8⁺ T cells over CD4⁺ T cells, while the situation is reversed in the presence of IL-7 and IL-15. However, DMSO does not have an impact on the subset distribution. Furthermore, DMSO does not alter the expression of activation and exhaustion markers (CD25, CD69 and PD-1 in FIG. 15) at concentrations up to 0.6%. At the highest DMSO concentration (1.2%), cell surface marker expression is increased. This increase seems to be more pronounced on CD4⁺ T cells. However, this corresponds to only a minor increase in cell surface expression levels as shown in the representative histograms in FIG. 15C, and FIG. 17.

Most importantly, we were able to validate that also in this CD3⁺ T cell expansion protocol, DMSO skews CD8⁺ T cell differentiation towards a memory phenotype, irrespective of the cytokine combination used (FIGS. 16 and 18). This skew is more pronounced than in the previous results where CD8⁺ T cells were cultured alone, as we now see an increase in the T_{CM} population from about 20% in the untreated condition to over 80% in the 1.2% DMSO condition. In contrast, the impact of DMSO on CD4⁺ T cell differentiation was less pronounced (FIG. 16), most probably because the majority of CD4⁺ T cells was already in a T_{CM} differentiation state in the absence of DMSO.

Furthermore, a DMSO concentration of 1.2% did not impact CD3⁺ T cell survival nor CD4′/CD8′ subset distribution (FIG. 17A). The frequency of cells that are CD25 and CD69 positive increases in the presence of DMSO (FIG. 17B). The effect for CD25⁺ seems to be more pronounced on CD8⁺ T cells while the effect for CD69 can be seen on both CD4⁺ and CD8⁺ T cell subtypes (FIG. 17C). Analysis of the exhaustion marker PD1 shows that the proportion of cells expressing PD-1 seems to slightly increase when cells are cultured in the presence of 1.2% DMSO (FIG. 17B-C). We could demonstrate that in this CD3⁺ T cell expansion protocol, DMSO skews CD3⁺ T cell differentiation towards a central memory phenotype (FIG. 18A). This skew is more pronounced in the CD4⁺ T cell subpopulation as compared to the CD8⁺ T cell subpopulation in which only minor changes are observed (FIG. 18B). In parallel, CD8⁺ T cells were purified from the same donor and cultured in the presence of DMSO. In the latter condition, CD8⁺ T cell differentiation was clearly skewed from naïve-like towards a T_{CM} phenotype, confirming earlier results described above (FIG. 18 A).

It is thus demonstrated herein that DMSO at low concentrations affects CD8⁺ T cell activation and differentiation in vitro. In contrast to what was observed before³⁵, no increase in cell death was apparent when T cells were cultured in the presence of low concentrations of DMSO for a longer period. Concentrations up to 1.2% can be used in human CD8⁺ T cultures in the presence of IL-2 or IL-7/-15 without hampering survival. In the presence of IL-2, concentrations up to 0.6% DMSO do not have an impact on proliferation either, which is in contrast to what was previously observed when CD4⁺ T cells are cultured in the presence of low concentrations of DMSO²⁹. Importantly, in the typical practice of total CD3⁺ T cell cultivation as is used in CAR-T cell manufacturing, concentrations of 1.2% DMSO allowed full viability.

Most importantly, we demonstrate that addition of DMSO to in vitro T cell cultures skews the CD8⁺ T cell differentiation state towards a memory phenotype. In particular, a decrease in CD45RA expression is observed in the presence of DMSO. CD45 is a tyrosine phosphatase that is alternatively spliced to generate isoforms of different molecular weights which are differentially expressed on T cells depending on their differentiation state. CD45RA⁺ T cells have naive characteristics while CD45RO⁺ (CD45RA⁻) T cells are considered memory T cells because they proliferate in response to recall antigens. Further, a role has been assigned to CD45RA in regulating the cell cycle leading to mitosis. As such, CD45RA expression has been shown to be low during the G0 and G1 stages of the cell cycle³⁶. In previous studies, where the effect of DMSO on stem cell differentiation was evaluated, it was shown that DMSO increases the time cells spend in the G1 phase of the cell cycle³⁰. We therefore hypothesize that the effect of DMSO on the cell cycle of T cells is reflected in a lower expression of CD45RA which is further associated with a memory-like phenotype.

It was previously reported that DMSO concentrations ranging from 2.5% to 10% inhibit cytokine production from CD4⁺ and CD8⁺ T lymphocyte subsets²⁴. These results are in agreement with another study demonstrating that long-term exposure to DMSO abolished both CD4⁺ and CD8⁺ T-lymphocyte antigen-specific responses³⁵. However, we studied the effect of DMSO at lower concentrations than the ones reported. Similarly, Avelar-Freitas et al observed that concentrations up to 1% v/v of DMSO did not change IFN-γ and TNF-α production in the cytoplasm of human lymphocytes and neutrophils stimulated with PMA for 4 hours³⁷. It was already shown that DMSO elicits a biphasic response as it promotes the secretion of pro-inflammatory cytokines such as IL-6 at low concentrations while inhibiting it at high concentrations^{26,38}. Other studies show that lymphoma cell death is prevented at DMSO concentrations of 1-2% while higher concentrations induce apoptosis³⁹. Moreover, it is not possible to directly compare the results from these studies with our study because of the different stimulation conditions, DMSO concentrations and incubation times. And, in these studies the T cell differentiation phenotype was not studied in detail.

Evaluation of the influence of DMSO on functional T cell responses in vitro may be done by studying the production profile of pro-inflammatory cytokines upon stimulation of cells. Furthermore, RNA sequencing experiments can be performed on activated T cells to identify differentially regulated transcriptional programs in cells cultured in the presence of DMSO.

Immunotherapies based on reinfusion of autologous cells incubated ex vivo with peptides reconstituted in solvents such as DMSO are now performed on a routine basis. Furthermore, therapeutic cell preparations such as the FDA approved CD19 CAR-T cell products Kymriah and Yescarta contain DMSO used as cryoprotectant. Hence, the molecule clearly is compliant with pharmaceutical use in the intended setting. However, the effect of DMSO on T cell responses has not been evaluated in detail. Our observation that either CD3⁺ T cells or purified CD8+ T cells can be skewed towards a memory phenotype can be advantageous when preparing T cells for adoptive transfer, since it has been described that the T cell differentiation state can have a major impact on the induction of antitumor immunity. In contrast to effector cells, memory cells have enhanced survival and proliferative potential, and the potential to provide more robust and enduring immune response against tumors⁴⁰. Adoptive cell transfer (ACT) of purified naive, stem cell memory and central memory T cell subsets results in superior persistence and antitumor immunity compared to ACT of populations containing more differentiated T cell subsets such as effector T cells and effector memory T cells⁴¹. The findings disclosed herein may thus have important implications for future cell-based immunotherapies. Addition of DMSO to T cell cultures for ACT, such as chimeric antigen receptor T (CAR-T) cells, may impact their antitumor efficacy in vivo.

### Materials and Methods

### Ethical Approval

All experiments were approved and performed according to the guidelines of the ethical committee Medical Ethics of Ghent University, Belgium. The breeding of NSG mice is approved by the ethical committee with file number E-726 and animal experiments are approved by the ethical committee with file number EC2020-009.

### Cell Lines

THP-1 cells were obtained from ATCC and cultured in RPMI medium (Gibco) supplemented with 10% FCS, 0.03% L-Gln, 0.4 mM sodium pyruvate and 50 μM β-mercaptoethanol. SKOV-3 cells expressing luciferase were kindly provided by Prof. De Wever (Ghent University, Faculty of Medicine and Health Sciences) and were cultured in DMEM medium (Gibco) supplemented with 10% FCS and 1% penicillin/streptomycin. Jurkat cells were obtained from ATCC and were cultured in RPMI medium (Gibco) supplemented with 10% FCS, 2 mM L-Gln and 0.4 mM sodium pyruvate. All cell lines were maintained in a 37° C., 5% CO₂, fully humidified incubator and passaged twice weekly.

### Guide RNA

We designed gRNA for the gene of interest using the Synthego design tool, and the sequence is depicted in SEQ ID NO:1 (https://www.synthego.com/products/bioinformatics/crispr-design-tool). GuideRNA was ordered as chemically modified synthetic sgRNA (with 2′O-Methyl at 3 first and 3 last bases and 3′ phosphorothioate bonds between first 3 and last 2 bases) and reconstituted at 100 μM in TE buffer. Two-part crRNA and tracrRNA were acquired from IDT and annealed following the recommended protocols in the datasheet. Briefly crRNA and tracrRNA were dissolved in TE buffer to a concentration of 80 μM, mixed at a molar ratio of 1:1 and incubated at 37° C. for 30 mins to obtain 40 μM hybrid gRNAs.

### RNP Electroporation

Recombinant Cas9-GFP protein was purchased from the VIB protein core (https://vib.be/labs/vib-protein-core). Cas9 RNP was made by incubating Cas9 protein with sgRNA at a molar ratio of 1:2 at 37° C. for 15 min immediately prior to electroporation in T cells. Electroporation was performed using the Lonza Amaxa 4D Nucleofector X unit (Program EH-115) and the P3 primary cell kit with the following conditions: 1×10⁶ cells/20 μL P3 buffer per cuvette (16-well strips) with 20 μM Cas9-RNP. Following nucleofection, 80 μL pre-warmed medium was added per well and cells were allowed to rest for 30 mins at 37° C., 5% CO₂.

### Analysis of Genome Editing Efficiency

0.1×10⁶ cells were collected and lysed in QuickExtract™ (Lucigen Epicentre) according to the supplier's instructions. The target site was amplified by PCR using FW and reverse primers as depicted in SEQ ID NO:2 and SEQ ID NO:3, respectively, and Sanger Sequenced using the forward primer. Sequencing data was analyzed with the ICE tool (Inference of CRISPR Edits, Synthego) to infer the percentage of insertions and deletions (INDEL score) and the percentage of insertions and deletions that are out of frame (knock out (KO) score)(13).

### Production of Retroviral Vectors

Retroviral constructs encoding the nanoCAR sequences were previously cloned in the LZRS-IRES-eGFP vector and were obtained from Prof. Dr. Bart Vandekerckhove (Department of Clinical Chemistry, Microbiology and Immunology, Ghent University, 9000 Ghent, Belgium). Viral particles were produced using standard Ca₃(PO₄)₂ transfection of the Phoenix ampho packaging cell line. Retroviral supernatant was collected at day 14 after transfection and puromycin selection and kept at −80° C. until use.

### Generation of NanoCAR Expressing Human T Cells

Immunocult-stimulated human CD3⁺ T cells were retrovirally transduced on Retronectin coated plates (TaKaRa). 500 μL of cells per well at 0.5×10⁶ cells/mL were supplemented with 0.5 mL retroviral supernatant and centrifuged for 90 minutes at 900 g at 32° C. Transduced cells were detected by eGFP expression or by an anti-VHH antibody directed against the nanobody constituting the extracellular domain of the CAR and analyzed by flow cytometry.

### Lectin-Based Flow Cytometry

Lectins were ordered at Vector laboratories. 2×10⁵ cells per condition were collected and rinsed three times with PBS. Cells were incubated with fixable viability dye eFI780 and biotinylated lectin in lectin binding buffer (PBS with 0.1 mM CaCl₂) for 30 minutes at 4° C. Competitive inhibitors were incubated with the respective lectins at least 30 minutes prior to staining. After rinsing with lectin binding buffer, cells were incubated with PE-coupled neutravidin (5 μg/mL) for 30 minutes at 4° C. After rinsing the cells with PBS, samples were resuspended and analyzed by flow cytometry. A minimum of 50 000 events was recorded. In conditions for which cells need to be fixed prior to lectin staining, cells were incubated with 4% PFA for 30 minutes at room temperature following the viability staining.

### PNGaseF Digest

In order to prepare cell surface N-glycans for DNA-Sequencer Assisted Fluorophore Assisted Carbohydrate Electrophoresis (DSA FACE) analysis, 1×10⁶ cells were collected per condition and washed three times with PBS to reduce the presence of medium derived glycans. Cell culture medium was collected for N-glycan labeling. PNGaseF digest (0.125 IU/1×10⁶ cells, in-house production) was performed in 25 μL final volume in PBS for 2 hours at 37° C. Cells were removed by centrifugation (5 min at 300 g) and the supernatant was subjected to another centrifugation step (15 min at 15 000 rpm) to remove cell debris. The remaining liquid portion of the sample was stored at −20° C. until APTS labeling and DSA-FACE analysis.

### N-Glycan Analysis Using DSA-FACE

The remaining N-glycan samples were labelled by adding an equal volume (20 μL) of labeling mix consisting of a 1/1 v/v mix of 1M morpholine borane in 20% DMSO, 20% SDS and 4M Urea mixed with 350 mM APTS in 2.4M citric acid and 14% SDS immediately prior to labeling. The labeling reaction was incubated at 70° C. for 1 hour and allowed to cool down at 4° C. before purification. Size exclusion chromatography (Sephadex G-10 resin with an exclusion limit of 700 Da prepared in a 96-well setup in Multiscreen-Durapore plates) was performed twice to desalt the samples and to remove free unreacted APTS(14). The labeled glycans were then dried in a speedvac.

Purified labelled and dried N-glycans were resuspended in 10 μL ultrapure water and analyzed with capillary electrophoresis on an eight-capillary DNA sequencer (Applied Biosystems 3500 Genetic analyzer). A proprietary internal standard (GlyXera) was added to the samples to be able to align profiles from different samples. Samples were injected on a 50 cm capillary at 15 kV for 10 seconds, using POP7 polymer and 100 mM TAPS, pH 8.0, containing 1 mM EDTA as the running buffer. N-glycan profiles were analyzed through the Genemapper 6 software.

### Flow Cytometry Analysis

Flow cytometry analysis was performed on 0.2×10⁶ cells per sample collected in a 96-well V bottom plate. Cells were rinsed with FACS buffer (PBS containing 0.5% BSA and 2 mM EDTA) for 3 min at 300 g and incubated with Fc Receptor Blocking solution (Human TruStain FcX™, Biolegend) block for 10 minutes prior to cell surface staining with fluorescently labeled antibodies in Brilliant Stain buffer (BD Biosciences) for 30 minutes at 4° C.

For human T cell phenotyping, cells were labeled with fluorescent antibodies against human CD8, CD62L and CD45RA (BD Biosciences) and CD3, CD4, CD25, CD69, CD197 (CCR7), TIM-3 (CD366), CD152 (CTLA4) and CD279 (PD-1) (Biolegend). A Fixable dye eFluor™ 780 (eBioscience) was used to evaluate live/dead cells.

Data were acquired on a BD Symphony AS or FACS Symphony™ equipped with five lasers (355, 405, 488, 561, 640 nm) (BD Biosciences) and analyzed using FlowJo software (Tree Star, Ashland, OR). Flow cytometer calibration was performed using CS&T beads (BD Biosciences). The gating strategy was set based on fluorescence minus one (FMO) controls and retained for all samples. Jurkat, THP-1 and SKOV-3 cell lines and primary human T cells were labeled with fluorescent antibody against human CD70 or isotype control (Biolegend) to verify antigen expression as described before(15).

Galectin expression by tumor cell lines was evaluated by cell surface staining with a fluorescent antibody against Galectin-3 and an antibody against Galectin-1. The latter was detected by a fluorescent anti-goat antibody. As a positive control, cells were incubated with 200 μg/mL recombinant Galectin-1 and Galectin-3 (Biolegend). Galectin binding was competitively inhibited by adding 50 mM lactose during the staining procedure.

In all analyses, following doublet exclusion, live cells were identified using a fixable viability dye (Molecular Probe, Life Technologies). Data were acquired on a BD Symphony A5 equipped with five lasers (355, 405, 488, 561, 640 nm) (BD Biosciences) and analyzed using FlowJo software (Tree Star, Ashland, OR).

### In Vitro Analysis of Cytokine Production

Glyco-engineered CD70 nanoCAR-T cells were stimulated in vitro by co-incubation with THP-1 or SKOV-3 tumor cell lines expressing CD70 at x cells in a 96-well plate in duplicate. After 1 hour of co-incubation, BD GolgiPlug (BD Biosciences) was added and after an additional 15 hours of stimulation, the cells were harvested, labelled with anti-CD3 #BV510, CD4 #AF700 and CD8 #BUV805, fixed and permeabilized (eBioscience) and labelled for intracellular expression cytokines with anti-TNF-α#BUV395 (BD Biosciences), IFN-γ#BV711 and IL-2 #PE (Biolegend). Samples were analyzed by flow cytometry on a BD Symphony AS equipped with five lasers (355, 405, 488, 561 and 640 nm) and data was analyzed using FlowJo software (Tree Star, Ashland, OR).

### In Vitro Analysis of Tumor Cell Killing

Glyco-engineered CD70 nanoCAR-T cells were incubated with 2×10⁴ THP-1 cells at different effector/target ratios (0; 0.0015; 0.015 and 0.15) in IMDM medium with Glutamax (Gibco) containing 10% FCS and 1% penicillin/streptomycin. Cells were labelled with fluorescent antibodies against CD3, CD4 and CD8 for the analysis of T cells and CD33 for the analysis of THP-1 cells at the start of the co-culture (day 0) and at day 3, 7, 10 and 14. At day 7 of co-culture, 2×10⁴ THP-1 cells were added to the remaining wells. Cell numbers were determined by flow cytometry.

In Vivo Analysis of Glyco-Engineered CD70 nanoCAR-T Cell Efficacy

NSG mice (breeder pairs obtained from The Jackson Laboratory, breeding in house) between 8-12 weeks of age were subcutaneously (in the flank) injected with 2×10⁶ SKOV-3 cells in 50 μL. The cells were allowed to form a solid mass and CD70 nanoCAR-T cells were intravenously injected on day 13 (in 200 μL total volume in PBS). Body weight and tumor progression was followed up by caliper and BLI. Hereto, a dose of 150 mg/kg D-luciferin potassium salt (Perkin Elmer) was injected intraperitoneally 10 minutes before imaging. BLI data were analyzed using Living Image Software and reported as photons/second.

### End-Point Analysis on Spleen and Blood

At day 34, mice were euthanized. Peripheral blood was collected following severing of the right atrium of the heart and transferred to EDTA coated Microvettes (Sarstedt). The volume of blood was determined and red blood cells were removed using ammonium-chloride-potassium lysis buffer (Lonza) prior to antibody staining for flow cytometry analysis.

The spleen was collected and processed to a cell suspension through a 70 μM cell strainer. Erythrocytes were removed using ammonium-chloride-potassium lysis buffer (Lonza) followed by washes. Cells were counted prior to antibody staining for flow cytometry analysis.

Tumors were isolated from non-treated controls and fixed in 4% PFA. Subsequently, tumor tissue was embedded in paraffin for downstream immunohistochemistry analysis.

### Immunohistochemistry and Microscopic Analysis of Galectin Expression in Tumor Tissue

Immunofluorescent staining was performed on 4 μm thick formalin-fixed, paraffin embedded (FFPE) sections of tumor samples from untreated mice. After antigen retrieval using citrate buffer pH 6 (Vector, H-3300), sections were incubated with 1% goat serum in PBS+0.5% BSA+0.1% Tween20 for 30 minutes to block non-specific binding. Subsequently, monoclonal rabbit anti-galectin-1 (1:200, Cell Signaling, Ref 13888S) or monoclonal rabbit anti-galectin-3 (1:200, Cell Signaling, Ref 87985S) diluted in 1% w/v goat serum in PBS+0.5% BSA+0.1% Tween₂₀ were incubated at overnight at 4° C. Alexa Fluor 568 labelled goat anti-rabbit (1:500, Thermofisher, A11036), was incubated at room temperature for two hours. Counterstaining was performed using DAPI (1:1000). Slides were mounted using 1% n-propyl-gallate in glycerol (pH7).

Images of the galectin staining were acquired with a LSM880 confocal microscope (Zeiss) and analyzed through ZEN Microscopy Software (Zeiss).

### Human CD8⁺ T Cell Isolation and Culture Under DMSO Exposure

Leukocyte-enriched buffy coat samples were obtained from healthy donors attending the Red Cross center after informed consent and ethical committee approval (EC2019-1083). Peripheral blood lymphocytes were prepared by a Ficoll-Paque density centrifugation as described in the instruction manual for Leucosep™ (Greiner bio-one). CD8⁺ T cells were isolated by negative selection (MojoSort™ Human CD8 T cell Isolation kit, Biolegend) according to the manufacture's protocol. Cells were cultured in RPMI 1640 medium (Gibco-BRL) supplemented with L-glutamine (0.03%, Gibco), 0.4 mM sodiumpyruvate (Merck Millipore) and 50 μM β-mercapto-ethanol (Sigma Aldrich) and 10% heat-inactivated fetal calf serum (FCS), in 48 well plates (Sarstedt) and stimulated with human anti-CD3/CD28 Dynabeads (1 bead:3 cell ratio) (Thermo Fisher Scientific) for 4 days at 37° C. Prior to cell seeding, Dynabeads and bead-bound cells were removed by separation on a magnet (after transfer to an Eppendorf tube) and the supernatant containing the CD8⁺ T cells was collected. Cells were washed twice with PBS before putting them in culture with different cytokine combinations: (1) recombinant human (rh) IL-2 at 50 units/mL (Life Technologies); (2) rhIL-7 at 10 ng/mL (R&D systems) and rhIL-15 at 1 ng/mL (Miltenyi). Cytokines and medium were replaced every 2-3 days. Where indicated, increasing concentrations of DMSO (Sigma) were added as given by % v/v.

### Human CD3⁺ T Cell Isolation and Culture Under DMSO Exposure

Leukocyte-enriched buffy coat samples were obtained from healthy donors attending the Red Cross center after informed consent and ethical committee approval (EC2019-1083). Peripheral blood lymphocytes were prepared by a Ficoll-Paque density centrifugation as described in the instruction manual for Leucosep™ (Greiner bio-one). CD3⁺ T cells were isolated by negative selection (MojoSort™ Human CD3 T cell selection kit, Biolegend) according to the manufacture's protocol. Cells were cultured in IMDM+Glutamax medium (Gibco-BRL) supplemented with 10% heat-inactivated fetal calf serum (FCS) and stimulated with Immunocult™ Human CD3/CD28 T-Cell Activator (Stemcell Technologies) (25 μL/10⁶ cells) for 3 days at 37° C. in the presence of 10 ng/mL IL-12 (Biolegend). Prior to cell seeding, cells were washed twice with PBS before putting them in culture with different cytokine combinations: (1) recombinant human (rh) IL-2 at 50 units/mL (Miltenyi); (2) rhIL-7 at 10 ng/mL (Miltenyi) and rhIL-15 at 10 ng/mL (Miltenyi). Cytokines and medium were replaced every 2-3 days. Cell densities were maintained between 1×10⁶ and 3×10⁶ cells/mL. Where indicated, increasing concentrations of DMSO (Sigma) were added as given by % v/v.

### Proliferation Assay

Human T cells were stained with Tag-it Violet™ Proliferation and Cell Tracking Dye (Biolegend), according to manufacturer's protocol. Tag-it Violet™ passively diffuses into the cell where esterases cleave acetoxymethyl esters (AM) on the molecule, Tag-it Violet™ then covalently attaches to intracellular proteins enabling its long-term retention.

### Statistical Analyses

### Multinomial Logistic Regression

We analysed the distributions of outcomes for the primary and secondary tumors in the different groups. To do so, we first had to define several possible outcomes. For the primary tumor, there are four possible outcomes: —Full control of the tumor, meaning that the tumor becomes undetectable both by caliper measurement and on BLI, and also no relapse. —Full control of the tumor but with a relapse after a period of the tumor being undetectable. —Partial control, meaning that the tumor stops growing but remains detectable, all these mice also had a relapse. —No control of tumor meaning that the tumor continually keeps growing. For the secondary tumor we only have full control, partial control or no control. The follow up time was not long enough to also consider relapses. To analyse these data in R⁴⁹, we used multinomial logistic regression (with a proportional odds assumption) as implemented in the polar function of the MASS package⁵⁰. We analysed the outcomes of experiments A and B together and so we had experiment as an additional predictor apart from group. Using likelihood ratio testing, we tested for an interaction effect between experiment and group and found that this was not significant in the primary nor secondary tumor. We used the multcomp package⁵¹ to calculate contrast estimates with 95% confidence intervals. We also used the ggpredict function from the ggeffects packages⁵² to calculate experiment-wise predictions with 95% confidence intervals for the predicted outcomes.

### Survival Analyses (Time to Relapse)

To analyze the time to relapse, we first defined the start of follow up as the moment the primary tumor was controlled or partially controlled. We define control as the first day the tumor became completely undetectable on BLI and by caliper measurement. We define partial control as the first day a tumor (that never fully disappears) stopped increasing in size according to caliper measurements. Next, we define a relapse event as the moment a tumor starts growing again. We take the last day before the tumor has increased in size again or became detectable again as the onset of relapse. The time to event is then the time between start of follow up and a relapse event and the follow up time is the time between start of follow up and either an event or the end of follow up in case of no relapse. We used R⁴⁹ with the survival^{53,54} and survminer⁵⁵ packages to generate Kaplan-Meier plots with estimates of the median survival times and a corresponding risk and events table. Since relapses were only observed in experiment B, we ran a straightforward analysis with group as the only predictor (groups: CD70 nanoCAR or CD70 nanoCAR-MGAT5 KO). We tested for the difference in survival probability in these groups with a logrank test as implemented in the survival package.

### Longitudinal Analyses

Tumor volumes were measured by measuring the length and width of a tumor and using the length*width*width/2 (this is a half cube or cuboid) approximation of the volume of a sphere. The minimal tumor volume that can be measured in this way is 0.5*0.5*0.5/2=0.0625 cm³ (which can be regarded as the limit of quantification). The smallest volume that can be reliable measured is about 0.5 cm³. We also cross-checked with BLI data for the small tumors, since this gives a better indication on whether there actually is still a tumor present or not. Whenever a small tumor was measured or a zero volume was registered, BLI was used to verify whether a tumor was actually present or not and the caliper measurements were adapted accordingly: when no tumor was found on BLI, we set small caliper measurements to zero and when a tumor was found on BLI but not measured by caliper, we set the tumor volume to 0.5. Uncontrolled tumor growth is exponential so we log-transformed (with a base 2 log) all tumor volume data to simplify the mean structures of the fitted models and to correct for the mean-variance relationship we observed during data exploration. To avoid problems when the tumor volume is zero, we first added 0.0625 (the detection limit) to all volumes before log-transforming. We then analyzed the transformed data of each experiment (A and B) and each phase (primary tumor before and after rechallenge and secondary tumor) separately by fitting a linear mixed model to each using the Ime4 package⁵⁵ and the nlminb fitting algorithm from the optimx package⁵⁶ in R⁴⁹. Where needed, we used piece-wise linear models with up to two knots for the time variable to allow for changes in growth rate over time. Random effects included a per-mouse random intercept and one or more random slopes for the time variable to model within-mouse correlation over time. For each model, we started with mean and covariance structures that were as saturated as possible based on the available data. Pruning the models was done via likelihood ratio testing first using Residual maximum likelihood (REML) to test for the random effects and then maximum likelihood (ML) to test for fixed effects. The final models were fitted using REML. In all models, we observed residual heteroscedasticity, even with the log-transformed data, so we used robust covariance estimators from the clubSandwich package⁵⁷ (vcovCR, type ‘CR0’) in conjunction with the multcomp package⁵¹ to calculate adjusted p-values and/or adjusted 95% confidence intervals for parameters and contrasts.

### SEQUENCE LISTING

>SEQ ID NO: 1: guide nucleotide sequence to
target MGAT5
GTGACTTTTGGCTTCATTTG
>SEQ ID NO: 2: Forward primer MGAT5
TCACAGCAGAATGGAAGT
>SEQ ID NO: 3: Reverse primer MGAT5
ACTGCTTATGAAGGCAGTGG
>SEQ ID NO: 4: mock (scrambled sgRNA not
targeting any gene):
GCACTACCAGAGCTAACTCA

### REFERENCES

1. De Bousser E, Meuris L, Callewaert N, Festjens N. Human T cell glycosylation and implications on immune therapy for cancer. Hum Vaccines Immunother 2020; 16, (10), 2374-2388.
2. Demotte N, Wieërs G, Van Der Smissen P, Moser M, Schmidt C, Thielemans K, Squifflet J-L, Weynand B, Carrasco J, Lurquin C, et al. A galectin-3 ligand corrects the impaired function of human CD4 and CD8 tumor-infiltrating lymphocytes and favors tumor rejection in mice. Cancer Res 2010; 70:7476-88.
3. Demetriou M, Granovsky M, Quaggin S, Dennis J W. Negative regulation of T-cell activation and autoimmunity by MGAT5 N-glycosylation. Nature 2001; 409:733-9.
4. Maletzki C, Bock S, Fruh P, Macius K, Witt A, Prall F, Linnebacher M. NSG mice as hosts for oncological precision medicine. Lab Invest 2019; 1-11.
5. Cedeno-Laurent F, Opperman M, Barthel S R, Hays D, Schatton T_{N}, Zhan Q, He X, Matta K L, Supko J G, Frank M H, et al. Metabolic inhibition of galectin-1-binding carbohydrates accentuates anti-tumor immunity. J Invest Dermatol 2012; 132:410-20.
6. Ye L, Park J J, Dong M B, Yang Q, Chow R D, Peng L, Du Y, Guo J, Dai X, Wang G, et al. In vivo CRISPR screening in CD8 T cells with AAV—Sleeping Beauty hybrid vectors identifies membrane targets for improving immunotherapy for glioblastoma. Nat Biotechnol 2019; 37:1302-13.
7. Sasawatari S, Okamoto Y, Kumanogoh A, Toyofuku T. Blockade of N-Glycosylation Promotes Antitumor Immune Response of T Cells. The Journal of Immunology 2020; 204:1373-85.
8. Rettinger E, Meyer V, Kreyenberg H, Volk A, Kuçi S, Willasch A, Koscielniak E, Fulda S, Wels W S, Boenig H, et al. Cytotoxic Capacity of IL-15-Stimulated Cytokine-Induced Killer Cells Against Human Acute Myeloid Leukemia and Rhabdomyosarcoma in Humanized Preclinical Mouse Models. Front Oncol 2012; 2.
9. Huey D D, Niewiesk S. Production of Humanized Mice Through Stem Cell Transfer. Current Protocols in Mouse Biology 2018; 8:17-27.
10. Collins A T, Lang S H. A systematic review of the validity of patient derived xenograft (PDX) models: the implications for translational research and personalised medicine. PeerJ 2018; 6.
11. Tatari N, Maich W T, Salim S K, Mckenna D, Venugopal C, Singh S. Preclinical Testing of CAR-T Cells in a Patient-Derived Xenograft Model of Glioblastoma. STAR Protocols 2020; 1:100174.
12. Hsiau T, Conant D, Rossi N, Maures T, Waite K, Yang J, Joshi S, Kelso R, Holden K, Enzmann B L, et al. Inference of CRISPR Edits from Sanger Trace Data. 2019.
13. Laroy W, Contreras R, Callewaert N. Glycome mapping on DNA sequencing equipment. Nat Protoc 2006; 1:397-405.
14. Yang M, Tang X, Zhang Z, Gu L, Wei H, Zhao S, Zhong K, Mu M, Huang C, Jiang C, et al. Tandem CAR-T cells targeting CD70 and B7-H3 exhibit potent preclinical activity against multiple solid tumors. Theranostics 2020; 10:7622-34.
15. Capriotti K, Capriotti J A. Dimethyl sulfoxide: History, chemistry, and clinical utility in dermatology. J Clin Aesthetic Dermatol 2012; 5:24-6.
16. David N A. The pharmacology of dimethyl sulfoxide. Annu Rev Pharmacol 1972; 12:353-74.
17. De La Vega F M, Mendoza-Figueroa T. Dimethyl sulfoxide enhances lipid synthesis and secretion by long-term cultures of adult rat hepatocytes. Biochimie 1991; 73:621-4.
18. Hollebeeck S, Raas T, Piront N, Schneider Y-J, Toussaint O, Larondelle Y, During A. Dimethyl sulfoxide (DMSO) attenuates the inflammatory response in the in vitro intestinal Caco-2 cell model. Toxicol Lett 2011; 206:268-75.
19. Pal R, Mamidi M K, Das A K, Bhonde R. Diverse effects of dimethyl sulfoxide (DMSO) on the differentiation potential of human embryonic stem cells. Arch Toxicol 2012; 86:651-61.
20. Thaler R, Spitzer S, Karlic H, Klaushofer K, Varga F. DMSO is a strong inducer of DNA hydroxymethylation in pre-osteoblastic MC3T3-E1 cells. Epigenetics 2012; 7:635-51.
21. Czysz K, Minger S, Thomas N. DMSO Efficiently Down Regulates Pluripotency Genes in Human Embryonic Stem Cells during Definitive Endoderm Derivation and Increases the Proficiency of Hepatic Differentiation. PLoS ONE 2015; 10.
22. Awan M, Buriak I, Fleck R, Fuller B, Goltsev A, Kerby J, Lowdell M, Mericka P, Petrenko A, Petrenko Y, et al. Dimethyl sulfoxide: a central player since the dawn of cryobiology, is efficacy balanced by toxicity?Regen Med 2020; 15:1463-91.
23. Yi X, Liu M, Luo Q, Zhuo H, Cao H, Wang J, Han Y. Toxic effects of dimethyl sulfoxide on red blood cells, platelets, and vascular endothelial cells in vitro. FEBS Open Bio 2017; 7:485.
24. de Abreu Costa L, Henrique Fernandes Ottoni M, dos Santos M G, Meireles A B, Gomes de Almeida V, de Fátima Pereira W, Alves de Avelar-Freitas B, Eustáquio Alvim Brito-Melo G. Dimethyl Sulfoxide (DMSO) Decreases Cell Proliferation and TNF-α, IFN-γ, and IL-2 Cytokines Production in Cultures of Peripheral Blood Lymphocytes. Mol J Synth Chem Nat Prod Chem 2017; 22.
25. Notman R, Noro M, O'Malley B, Anwar J. Molecular Basis for Dimethylsulfoxide (DMSO) Action on Lipid Membranes. J Am Chem Soc 2006; 128:13982-3.
26. Elisia I, Nakamura H, Lam V, Hofs E, Cederberg R, Cait J, Hughes M R, Lee L, Jia W, Adomat H H, et al. DMSO Represses Inflammatory Cytokine Production from Human Blood Cells and Reduces Autoimmune Arthritis. PLoS ONE 2016; 11.
27. Lin G-J, Sytwu H-K, Yu J-C, Chen Y-W, Kuo Y-L, Yu C-C, Chang H-M, Chan D-C, Huang S-H. Dimethyl sulfoxide inhibits spontaneous diabetes and autoimmune recurrence in non-obese diabetic mice by inducing differentiation of regulatory T cells. Toxicol Appl Pharmacol 2015; 282:207-14.
28. Huang S S, Chen C-L, Huang F W, Hou W-H, Huang J S. DMSO Enhances TGF-β Activity by Recruiting the Type II TGF-β Receptor From Intracellular Vesicles to the Plasma Membrane. J Cell Biochem 2016; 117:1568-79.
29. Holthaus L, Lamp D, Gavrisan A, Sharma V, Ziegler A-G, Jastroch M, Bonifacio E. CD4+ T cell activation, function, and metabolism are inhibited by low concentrations of DMSO. J Immunol Methods 2018; 463:54-60.
30. Chetty S, Pagliuca F W, Honore C, Kweudjeu A, Rezania A, Melton D A. A simple tool to improve pluripotent stem cell differentiation. Nat Methods 2013; 10:553-6.
31. Sambo D, Li J, Brickler T, Chetty S. Transient Treatment of Human Pluripotent Stem Cells with DMSO to Promote Differentiation. J Vis Exp JoVE 2019;
32. Sedwick C. Clearing the way for efficient stem cell differentiation. J Cell Biol 2015; 210:1036-1036.
33. Schoenberger S P. CD69 guides CD4+ T cells to the seat of memory. Proc Natl Acad Sci USA 2012; 109:8358-9.
34. Mujib S, Jones R B, Lo C, Aidarus N, Clayton K, Sakhdari A, Benko E, Kovacs C, Ostrowski M A. Antigen-Independent Induction of Tim-3 Expression on Human T Cells by the Common γ-Chain Cytokines IL-2, IL-7, IL-15, and IL-21 Is Associated with Proliferation and Is Dependent on the Phosphoinositide 3-Kinase Pathway. J Immunol 2012; 188:3745-56.
35. Kloverpris H, Fomsgaard A, Handley A, Ackland J, Sullivan M, Goulder P. Dimethyl sulfoxide (DMSO) exposure to human peripheral blood mononuclear cells (PBMCs) abolish T cell responses only in high concentrations and following coincubation for more than two hours. J Immunol Methods 2010; 356:70-8.
36. LaSalle J M, Hafler D A. The coexpression of CD45RA and CD45RO isoforms on T cells during the S/G2/M stages of cell cycle. Cell Immunol 1991; 138:197-206.
37. Avelar-Freitas B A, Almeida V G, Santos M G, Santos JAT, Barroso P R, Grael CFF, Gregorio L E, Rocha-Vieira E, Brito-Melo GEA. Essential oil from Ageratum fastigiatum reduces expression of the pro-inflammatory cytokine tumor necrosis factor-alpha in peripheral blood leukocytes subjected to in vitro stimulation with phorbol myristate acetate. Rev Bras Farmacogn 2015; 25:129-33.
38. DeForge L E, Preston A M, Takeuchi E, Kenney J, Boxer L A, Remick D G. Regulation of interleukin 8 gene expression by oxidant stress. J Biol Chem 1993; 268:25568-76.
39. Lin C K, Kalunta C I, Chen F S, Nguyen T T, Kaptein J S, Lad P M. Dimethyl sulfoxide suppresses apoptosis in Burkitt's lymphoma cells. Exp Cell Res 1995; 216:403-10.
40. Perret R, Ronchese F. Effector CD8+ T cells activated in vitro confer immediate and long-term tumor protection in vivo. Eur J Immunol 2008; 38:2886-95.
41. Klebanoff C A, Scott C D, Leonardi A J, Yamamoto T N, Cruz A C, Ouyang C, Ramaswamy M, Roychoudhuri R, Ji Y, Eil R L, et al. Memory T cell-driven differentiation of naive cells impairs adoptive immunotherapy. J Clin Invest 126:318-34.
42. Vairy S, Garcia J L, Teira P, Bittencourt H. CTL019 (tisagenlecleucel): CAR-T therapy for relapsed and refractory B-cell acute lymphoblastic leukemia. Drug Des Devel Ther 2018; 12:3885-98.
43. Reagan P M, Friedberg J W. Axicabtagene ciloleucel and brexucabtagene autoleucel in relapsed and refractory diffuse large B-cell and mantle cell lymphomas. Future Oncol Lond Engl 2021; 17:1269-83.
44. Okada M, Chikuma S, Kondo T, Hibino S, Machiyama H, Yokosuka T, Nakano M, Yoshimura A. Blockage of Core Fucosylation Reduces Cell-Surface Expression of PD-1 and Promotes Anti-tumor Immune Responses of T Cells. Cell Rep 2017; 20:1017-28.
45. Alegre M L, Frauwirth K A, Thompson C B. T-cell regulation by CD28 and CTLA-4. Nat Rev Immunol 2001; 1:220-8.
46. Lau K S, Partridge E A, Grigorian A, Silvescu C I, Reinhold V N, Demetriou M, Dennis J W. Complex N-Glycan Number and Degree of Branching Cooperate to Regulate Cell Proliferation and Differentiation. Cell 2007; 129:123-34.
47. Starr T K, Daniels M A, Lucido M M, Jameson S C, Hogquist K A. Thymocyte Sensitivity and Supramolecular Activation Cluster Formation Are Developmentally Regulated: A Partial Role for Sialylation. J Immunol 2003; 171:4512-20.
48. Goud N S, Soukya PSL, Ghouse M, Komal D, Alvala R, Alvala M. Human Galectin-1 and its inhibitors: Privileged target for cancer and HIV. Mini Rev Med Chem 2019; 19(16):1369-1378.
49. R Core Team, A Language and environment for statistical computing (R Foundation for Statistical Computing, Vienna, Austria, 2022; https://www.R-project.org/).
50. W. N. Venables, B. D. Ripley, Modern Applied Statistics with S. Fourth Edition (Springer, New York, 2002).
51. T. Hothorn, F. Bretz, P. Westfall, Simultaneous Inference in General Parametric Models.
52. Lüdecke D, ggefects: Tidy Data Frames of Marginal Effects from Regression Models (2018).
53. T. Therneau, A Package for Survival Analysis in R (2022; https://CRAN.R-project.org/package=survival).
54. T. Therneau, P. Grambsch, Modeling Survival Data: Extending the Cox Model (Springer, New York, 2000).
55. A. Kassambra, M. Kosinski, P. Biecek, Survminer: Drawing Survival Cuvres using “ggplot2” (2021; https://CRAN.R-project.org/package=survminer).
56. J. C. Nash, R. Varadhan, J. Stat. Softw. 43, 1-14 (2011).
57. J. Pustejovsky, ClubSandwich: Cluster-Robust (Sandwich) Variance Estimators with Small-Sample Corrections (2022; https://CRAN.R-project.org/package=clubSandwich).

1. A human CAR-T cell comprising a mutation in the N-acetylglucosaminyltransferase V (MGAT5) gene as compared to a wild type MGAT5 gene.

2. The MGAT5 mutated human CAR-T cell of claim 1, wherein the mutation is a knock-out mutation of MGAT5.

3. The MGAT5 mutated human CAR-T cell of claim 1, wherein the cell is devoid of tetra-antennary N-glycans at its cell surface.

4. A method of administering a CAR-T cell to a subject, the method comprising: administering the MGAT5 mutated human CAR-T cell of claim 1 to the subject.

5. The method according to claim 15, wherein the cancer is a haematological malignancy or solid tumor type of cancer.

6. The method according to claim 15, wherein administration of the CAR-T cell inhibits primary and/or secondary tumor growth and/or tumor burden in a subject.

7. The method according to claim 5, wherein the administration reduces the chance of cancer relapse in the subject.

8. A method of producing a MGAT5 mutated CAR-T cell composition with a predominant memory phenotype, the method comprising:
(a) isolating T cells from a primary sample and generating a T cell composition comprising at least 1×10⁶ T cells/mL,
(b) incubating the T cell composition under stimulating conditions comprising 0.3-1.2% (v/v) Dimethylsulfoixde (DMSO), thereby generating a stimulated T cell composition,
(c) engineering the stimulated T cell composition by contacting the stimulated T cells with an agent comprising a polynucleotide encoding a recombinant chimeric antigen receptor, and introducing a mutation in the MGAT5 gene,
(d) cultivating the stimulated T cell composition of step b) in a medium comprising 0.3-1.2% (v/v) DMSO for expansion of the stimulated T cell composition.

9. The method according to claim 8, wherein introducing the mutation in the MGAT5 gene comprises CRISPR/Cas engineering.

10. (canceled)

11. (canceled)

12. (canceled)

13. (canceled)

14. The human MGAT5 mutated CAR-T cell of claim 1, wherein the human MGAT5 mutated CAR-T cell is comprised in a pharmaceutical composition.

15. The method according to claim 4, wherein the subject is suffering from cancer.


SEQUENCE LISTING (4 sequences)

SEQ ID NO: 1 - RNA, 20 nt, synthetic construct
  source 1..20: mol_type = other RNA
  misc_feature 1..20: note = guide nucleotide sequence to target MGAT5
gtgacttttggcttcatttg

SEQ ID NO: 2 - DNA, 18 bp, synthetic construct
  source 1..18: mol_type = other DNA
  misc_feature 1..18: note = primer
tcacagcagaatggaagt

SEQ ID NO: 3 - DNA, 20 bp, synthetic construct
  source 1..20: mol_type = other DNA
  misc_feature 1..20: note = primer
actgcttatgaaggcagtgg

SEQ ID NO: 4 - RNA, 20 nt, synthetic construct
  source 1..20: mol_type = other RNA
  misc_feature 1..20: note = mock (scrambled sgRNA not targeting any gene)
gcactaccagagctaactca